(19) United States
(12) Patent Application Publication
(10) Pub. No.: US 2026/0286316 A1
(43) Pub. Date: Sep. 24, 2026

(54) METHOD OF PRODUCTION OF MODIFIED PHAGE AND METHOD OF TREATMENT USING SAME

(71) Applicants: The University of Adelaide, Adelaide (AU); AusHealth Corporate Pty Ltd, Underdale (AU)

(72) Inventors: Peter-John Wormald, North Adelaide (AU); Sarah Sylvia Joseph Vreugde, North Brighton (AU); Sha Liu, Woodville South (AU); Alkiviadis James Psaltis, Woodville South (AU); George Spyro Bouras, Joslin (AU); Keith Edward Shearwin, Adelaide (AU)

(21) Appl. No.: 18/881,218

(22) PCT Filed: Jul. 5, 2023
(86) PCT No.: PCT/AU2023/050622

§ 371 Date: Jan. 3, 2025

(30) Foreign Application Priority Data
Jul. 5, 2022 (AU) 2022901885

(51) Int. Cl. C12N 7/00; A61K 35/76; A61P 31/04

(52) U.S. Cl. CPC C12N 7/00; A61K 35/76; A61P 31/04; C12N 2795/00032; C12N 2795/00051

(57) ABSTRACT

A method of producing modified bacteriophage (“phage”) that are capable of infecting and causing lysis of a parent host bacteria that is insensitive to the unmodified phage is disclosed. The method comprises: treating a host bacteria insensitive to a selected phage strain with a suitable amount of the said phage strain and at least one antibacterial agent comprising protein synthesis inhibitory activity, and culturing the bacteria for a period and under conditions suitable for the phage to infect the bacteria and replicate to generate progeny phage; and recovering and expanding progeny phage which are phage with a modified capability to infect and cause lysis of said host bacteria. Methods of treatment involving the use of the modified phage, as well as pharmaceutical compositions comprising same are also disclosed.

### TECHNICAL FIELD

The present disclosure relates to the field of bacteriophage therapy, particularly in the circumstance of the treatment of a drug-resistant bacteria which is also bacteriophage non-sensitive (insensitive). Among other things, the present disclosure relates to a method of producing modified bacteriophage that are capable of infecting and causing lysis of a parent host bacteria that is insensitive to the unmodified bacteriophage.

### PRIORITY DOCUMENT

The present application claims priority from Australian Provisional Patent Application No. 2022901885 titled “METHOD OF PRODUCTION AND TREATMENT” and filed on 5 Jul. 2022, the content of which is hereby incorporated by reference in its entirety.

### BACKGROUND

Many human and animal illnesses are caused by infection with bacteria. The economic and social toll of bacterial disease is enormous. However, while the development of antibiotics has largely mitigated the effects of diseases caused by bacteria, the development of bacteria that are resistant to the action of antibiotics has ushered in an undesirable era where the suite of antibiotics for use has become more limited and made some bacterial infections untreatable as they have acquired resistance to the known antibiotics.

Indeed, the misuse and abuse of antibiotics, either among humans or among animals, has boosted the fast expansion of drug-resistant microorganisms. Current studies suggest that the mechanisms of bacterial antibiotic resistance include either a reduction in the affinity of the antibiotic agent to bind to its bacterial target protein or a reduction in the bacterial intracellular drug concentration. The former is primarily caused by the emergence of mutation(s) in the genes encoding the target protein of the antibiotic, while the latter is mainly achieved through alterations in the plasma membrane permeability, expression of degradation/modification enzymes, or overexpression of efflux pumps.

Additionally, bacteria can increase their tolerance and resistance to antibiotics through the formation of a biofilm, which then acts as a natural barrier to prevent the absorption of antibacterial drugs such as antibiotics. The biofilm drug-resistance mechanism is different from that of individual bacteria and, compared with planktonic bacteria, this drug-resistance mechanism can increase the bacterial antibiotic tolerance 10- to 1000-fold, and is one of the main causes of current bacterial drug resistance. The extracellular polymeric substance (EPS) secreted by bacteria, one of the main components of a biofilm, forms a dense and negatively charged natural barrier, which prevents the penetration of antibiotics and other antibacterial drugs into the membrane, thus reducing the concentration of drugs inside the membrane. Additionally, the lack of enough nutrients inside of the biofilm will inhibit the bacterial metabolic activity, and subsequently promote bacteria to enter a dormant state, making them more resistant to the drug during the reproduction period as many antibiotics rely on proliferation of the bacteria for effectiveness.

Moreover, the mechanisms for the resistance of bacteria to antibiotics and formation of biofilms are complex, and there are synergistic effects among the various mechanisms. Thus, drug-resistant bacteria, especially the so-called “superbugs” (eg methicillin-resistant Staphylococcus aureus (MRSA)), have rapidly spread to every corner of the world, leading to longer hospital stays, higher medical costs and mortality rates. Indeed, antibiotic-resistant bacteria now represent one of the greatest threats to global public health. Therefore, identifying and developing new ways to treat bacteria, including drug-resistant bacteria, quickly and effectively has become critical.

Bacteriophage (“phage”) can invade their bacteria host to disrupt the host metabolism and, eventually, cause lysis and killing of the host cell. Phage were first applied to anti-bacterial infection treatments in clinics over 100 years ago, and they were utilised specifically to kill and target bacterial hosts causing infection. Due to the discovery and widespread use of antibiotic agents, the use of phage has diminished. However, due to the ongoing problem of drug-resistant bacteria (and especially, the multidrug-resistant (MDR) bacteria), phage therapy has now returned to the spotlight. Further, it is now more fully appreciated that phage possess unique advantages that may mean that phage targeting specific pathogenic bacteria can be isolated to meet clinical needs more quickly than the development of novel antibacterial drugs. Phage are also very abundant in nature, lack cross-resistance with antibiotics, and are highly specific (compared to antibiotics). Moreover, phage can also be effective in disrupting bacterial biofilms, and offer enormous potential to serve as the basis of therapies providing an effective and sustainable solution for the treatment of bacteria, including treatment for superbugs and refractory MDR bacterial infections.

Notwithstanding the renewed interest in phage and phage therapy, the utility of phage in the clinic has been limited. Part of the reason for the lack of adoption of phage therapy is the narrow antibacterial spectrum of many phage. Further, many clinical isolates are resistant to known phage. In such conditions, a patient may have a considerable waiting period until a suitable phage is available for treating their specific bacterial infection and/or there are no phage suitable to infect a particular resistant bacterial infection. For this reason, strategies for phage therapy need to be identified which address the problem of the occurrence or development of phage insensitive bacteria. To this end, the Applicants looked to determine whether phage insensitive bacteria could be “re-sensitised” to phage and thereby be treatable by phage therapy.

### SUMMARY

According to a first aspect, the present disclosure provides a method of producing a modified phage, said method comprising the steps of:
(i) treating a host bacteria insensitive to a selected phage strain (ie a phage non-sensitive, or phage insensitive, or phage resistant bacteria) with a suitable amount of the said phage strain and at least one antibacterial agent comprising protein synthesis inhibitor activity, and culturing the bacteria for a period and under conditions suitable for the phage to infect the bacteria and replicate to generate progeny phage; and
(ii) recovering and expanding progeny phage which are phage with a modified capability to infect and cause lysis of said host bacteria.

In some embodiments, the host bacteria is a sample of a bacteria causative of an infection being suffered by a subject, and the phage is a phage selected from a “phage bank” and is one that is normally regarded as infectious for the host bacteria if not for the circumstance that the bacteria has become insensitive to the phage (ie is phage non-sensitive). The progeny phage have a modified capability to infect and cause lysis of said host bacteria, and may therefore be useful in treating the infection in the subject.

In a second aspect, the present disclosure provides a method of treating a bacterial infection in a subject, said method comprising the steps of:
1. obtaining a sample of a bacteria causative of an infection in said subject,
2. treating the bacteria with a suitable amount of a selected phage strain to which the bacteria is insensitive (ie the bacteria is phage non-sensitive) and at least one antibacterial agent comprising protein synthesis inhibitory activity, and culturing the bacteria for a period and under conditions suitable for the phage to infect the bacteria and replicate to generate progeny phage;
3. recovering and expanding progeny phage which are phage with a modified capability to infect and cause lysis of said host bacteria; and
4. administering to the subject an effective amount of the progeny phage, or a further propagated form thereof, to treat the infection.

In a third aspect, the present disclosure provides a modified phage produced in accordance with the method of the first aspect, or a further propagated form thereof.

In a fourth aspect, the present disclosure provides a pharmaceutical composition comprising a modified phage produced in accordance with the method of the first aspect, or a further propagated from thereof, optionally in combination with at least one pharmaceutically acceptable additive, carrier, diluent and/or excipient.

In a fifth aspect, the present disclosure provides the use of a modified phage produced in accordance with the method of the first aspect, or a further propagated form thereof, in the manufacture of a medicament for treating an infection in a subject.

In a sixth aspect, the present disclosure provides a modified phage, produced in accordance with the method of the first aspect, or a further propagated form thereof, for treating an infection in a subject.

In a seventh aspect, the present disclosure provides a method of killing bacteria, said method comprising contacting the bacteria with a modified phage produced in accordance with the method of the first aspect or a further propagated form thereof.

In an eighth aspect, the present disclosure provides an isolated phage lysin protein (an endolysin) derived from a modified phage produced in accordance with the method of the first aspect.

### BRIEF DESCRIPTION OF FIGURES

FIG. 1 provides the results of experimentation to determine any synergy between phage and various antibiotic agents on bacterial viability. Protein synthesis inhibitors (PSI) erythromycin (E), clindamycin (C) and azithromycin (Az), but not cell wall actives amoxicillin (A) and vancomycin (V), were found to have synergistic effects with phage J-Sa36 (Sa36), Sa83 and Sa87 to reduce or eradicate a clinical isolate of S. aureus (CI3). OD600 values, representing bacterial viability, were measured after 24 hours in the absence (TSB) or presence of ½ MIC (minimum inhibitory concentration) antibiotics alone, phage alone (MOI=0.2), or the combination of ½ MIC antibiotics and phage (MOI=0.2). Significant reductions were observed, compared to treatment with phage or antibiotic alone, for all combinations of phage and PSIs. n=3. Bars represent standard error of the mean (SEM). ns=not significant, ****, p<0.0001;

FIG. 2 provides the results of experiments to assess whether enhanced bacterial killing by the combination of clindamycin and phage was dose dependent. The results show that low (⅛^{th} MIC) doses of clindamycin in combination with J-Sa36 (Sa36) were effective against S. aureus (ATCC51650 and CI11). OD 600 values represent bacterial growth of (A) ATCC51650 and (B) CI11 after 24 hours exposure to 105 PFU/ml J-Sa36 (MOI=0.2) and varying clindamycin concentrations (0.005 μg/ml- 1/32 MIC to 0.1 μg/ml-½ MIC) or control (TSB). ns=not significant, ****, p<0.0001, one-way ANOVA with Tukey's multiple comparisons test. n=3. Bars represent standard error of the mean (SEM). Black bars represent S. aureus ATCC51650 and CI11 treated with medium (TSB), J-Sa36 (Sa36) or clindamycin (½ MIC or 0.1 μg/ml) as control. Grey bars represent S. aureus ATCC51650 and CI11 treated with J-Sa36 combined with different concentrations of clindamycin;

FIG. 3 provides results showing that the phage J-Sa36, Sa83 or Sa87 combined with clindamycin reduces the viability of ATCC51650 and CI11 biofilms. In particular, the reduction of biofilm viability of S. aureus (A) ATCC51650 and (B) CI11 was measured by Alamar Blue viability assay, and the measurements normalised and compared to untreated control (0 μg/ml). The concentrations of clindamycin ranged from 1, 0.5, 0.2, 0.1, 0.05 and 0 μg/ml. ns=not significant, *, p<0.05, Significance was determined by one-way ANOVA with Dunnett's multiple comparisons test in comparison with phage only controls. n=3. Bars represent standard error of the mean (SEM);

FIG. 4 provides results which show the time dependent effects on phage-antibiotic synergy: (A, B) S. aureus ATCC51650 or CI11 were grown in the absence (control) or presence of J-Sa36 (Sa36; MOI 0.2) or ½ MIC clindamycin for 3.5 hours, followed by the addition of ½ MIC clindamycin (Sa36+½ MIC clindamycin added at 3.5 hrs) or phage J-Sa36 (½ MIC clindamycin+J-Sa36 added at 3.5 hrs) with OD600 recorded at 3.5 (black bars) and 24 hours (grey bars); (C, D) ATCC51650 and CI11 respectively were pre-treated for different times with either ½ MIC clindamycin (black bar) or TSB only (grey) before J-Sa36 (MOI 0.2) was added at 1, 2, 3, 3.5, 4, 6 and 24 hrs. OD600 was recorded 24 hours later. n=3. ns=not significant, ****, p<0.0001; ***, p<0.001; **, p<0.01; *, p<0.05, mixed ANOVA with Tukey's multiple comparisons test. Bars represent standard error of the mean (SEM);

FIG. 5 provides the results of in vivo efficacy tests of phage and antibiotic combinations. Colony Forming Unit (CFU) counts (CFU/ml) (A) and biofilm biomass (μm³/μm²) (B) of a fresh piece of rat sinonasal mucosa from rats treated with 0.5×10¹⁰ PFU/ml Sa87 in combination with ½ MIC clindamycin [Sa87+C], 0.5×10¹⁰ PFU/ml Sa87 in combination with ½ MIC azithromycin [Sa87+Az], 0.5×10¹⁰ PFU/ml Sa87 [Sa87], ½ MIC clindamycin [C], ½ MIC azithromycin [Az], saline [Saline]. Experiments were performed with three replicates for each rat, averaged and each rat represented by 1 dot. ****, p<0.0001; **, p<0.01; *, p<0.05, One-way ANOVA with Dunnett's post hoc test. Error bars represent standard error of the mean (SEM).

FIG. 6 provides graphical results showing that the combination of S. aureus phage and ½ MIC clindamycin kills non-sensitive S. aureus strains: (A) S. aureus C43 treated with APTC-SA-2 and ½ MIC clindamycin; (B) S. aureus C330 treated with APTC-SA-2 and ½ MIC clindamycin; (C) S. aureus ATCC25923 treated with APTC-SA-2 and ½ MIC clindamycin; (D) S. aureus C285 treated with APTC-SA-12 and ½ MIC clindamycin; (E) S. aureus C319 treated with APTC-SA-12 and ½ MIC clindamycin; and (F) S. aureus ATCC25923 treated with APTC-SA-12 and ½ MIC clindamycin. Significance was determined compared to untreated control. Data expressed as mean±SEM for three independent experiments. ****, p<0.0001; ns, no significant. C=clindamycin;

FIG. 7 provides the results of bactericidal activity tests performed on exit phage. The exit phage's bactericidal activity was tested in its own host, S. aureus C43 (A), C330 (B), C285 (C) and C319 (D), each time treated with TB (control), the entry phages APTC-SA-2 (A, B) and APTC-SA-12 (C, D) (MOI=1) or exit phages APTC-SA-2-C43 (A), APTC-SA-2-C330 (B), APTC-SA-12-C285 (C) and APTC-SA-12-C319 (D). Significance was determined compared to untreated control. Data expressed as mean±SEM for three independent experiments. **, p<0.01; ****, p<0.0001; ns, no significant;

FIG. 8 shows graphical results of tests for the exit phage specificity and lytic activity. S. aureus clinical isolates C43 (A, E), C330 (B, F), C285 (C, G) and C319 (D, H) were treated with TSB (control), entry phages APTC-SA-2 (A, B) or APTC-SA-12 (C, D), or exit phages APTC-SA-2-C43 (A, E), APTC-SA-2-C330 (B, F), APTC-SA-12-C285 (C, G) or APTC-SA-12-C319 (D, H) at MOI=1 (A-D) or at MOI 0.2-0.001 (E-H). Significance was determined compared to untreated control. Data expressed as mean±SEM for three independent experiments. ****, p<0.0001; ns, not significant. ATCC=ATCC25923;

FIG. 9 provides characterising (stability and growth) results for entry phage and exit phage: (A) Entry phage APTC-SA-2 and APTC-SA-12 and exit phage APTC-SA-2-C43, APTC-SA-2-C330, APTC-SA-12-C285 and APTC-SA-12-C319 temperature stability was tested between 4 to 80° C.; (B) Entry phage APTC-SA-2 and APTC-SA-12 and exit phage APTC-SA-2-C43, APTC-SA-2-C330, APTC-SA-12-C285 and APTC-SA-12-C319 pH stability was tested between pH 3 to 12; (C) Entry phage APTC-SA-2 and APTC-SA-12 and exit phage APTC-SA-2-C43, APTC-SA-2-C330, APTC-SA-12-C285 and APTC-SA-12-C319 one-step growth curve were performed. Data expressed as mean±SEM for three independent experiments; and

FIG. 10 shows the results of a representative PHEARLESS Assay to determine the lytic activity of exit phage produced in accordance with the method of the disclosure. The expressed lysin protein (LysK) of the exit phage (APTC-SA-2-C330 (“294”) and APTC-SA-2-C319 (“298”)) was applied to S. aureus strains (C319) at the various concentrations indicated in the figure. “302” is a negative control (expressing no phage lysin). Arrow=lysis plaque.

### DETAILED DESCRIPTION

As described in the examples hereinafter, the Applicants found that treatment of phage non-sensitive S. aureus with lytic phage in combination with a sub-inhibitory concentration (MIC) of erythromycin, clindamycin and azithromycin, can significantly reduce the growth of the bacteria. Characterisation of the phage isolated after the treatment (“exit phage”) then, surprisingly, revealed that the phage were able to re-infect their “parent” bacterial host without the need (or “help”) of the antibiotic, and moreover, with up to a 1000-fold higher level of lytic activity as compared to that of the “entry phage” (ie the initial phage used). In other words, the dosage of exit phage to have significant antibacterial effects against the parent bacterial host was less than 1000^{th} the dosage of the entry phage and the entry phage could only infect the parent bacteria in the presence of protein synthesis inhibitor (PSI) antibiotics whilst the exit phage could infect the parent bacterial strains also in the absence of PSI antibiotics. The stability (ie temperature and pH stability) of the exit phage and their growth curve were, however, substantially unchanged and, in particular, showed no significant difference when compared with the corresponding entry phage. In addition, it was found that the burst size of the exit phage was higher than the entry phage (ie the burst size (calculated at 30 min) for entry phage APTC-SA-2 was 30 PFUs/infected cell; while for exit phage APTC-SA-2-43, the burst size was 40 PFUs/infected cell, and for exit phage APTC-SA-2-330, 42 PFUs/infected cell. Similarly, for entry phage APTC-SA-12, the burst size was 14 PFUs/infected cell, in comparison to burst sizes of 33 and 38 PFUs/infected cell, respectively, for the exit phage APTC-SA-12-C285 and exit phage APTC-SA-12-319). Moreover, from a bacteriophage insensitive mutant (BIM) frequency test, it was found that exit phage showed a significantly lower BIM frequency compared to entry phage. As such, the Applicants have identified a novel method for the production of a modified phage, derived from a phage for which a host bacterial strain is or has become insensitive (ie the bacteria is phage non-sensitive), which is capable of infecting the host bacteria strain and, preferably, also shows an enhanced level of lytic activity with a reduction in dosage required to achieve bacterial eradication, an increase in the burst size of the exit phage, and/or achieves a reduction in the frequency of emergence of bacteriophage insensitive mutants (BIM) when bacteria are treated with the exit phage compared to when they are treated with the parent entry phage (in the presence of sub-inhibitory PSI antibiotics).

Thus, in a first aspect, the present disclosure provides a method of producing a modified phage, said method comprising the steps of:
(i) treating a host bacteria insensitive to a selected phage strain (ie a phage non-sensitive, or phage insensitive, or phage resistant bacteria) with a suitable amount of the said phage strain and at least one antibacterial agent comprising protein synthesis inhibitor activity, and culturing the bacteria for a period and under conditions suitable for the phage to infect the bacteria and replicate to generate progeny phage; and
(ii) recovering and expanding progeny phage which are phage with a modified capability to infect and cause lysis of said host bacteria.

In this first aspect, the present disclosure provides a method of producing phage with improved lytic activity against a phage insensitive bacteria, by exposing the insensitive host bacterium to a selected infecting phage and an agent having protein synthesis inhibitory activity, and producing a phage with improved lytic activity against the insensitive bacteria.

The host bacteria used in step (i) may be any phage insensitive bacteria strain, but preferably, will be a phage insensitive bacteria strain of medical and/or veterinary significance.

In some embodiments, the host bacteria is selected from the group consisting of Staphylococcus species, Streptococcus species, Enterococcus species, Corynebacterium species, Bacteroides species, Bacillus species, Yersinia species, Legionella species, Francisella species, Salmonella species, Gordonia species, Mycobacterium species, Acinetobacter species, Clostridium species, Pseudomonas species, and Enterobacteriaceae species.

In some embodiments, the host bacteria is selected from the group consisting of S. aureus, S. epidermidis, S. lugdunensis, Acinetobacter baumannii, Clostridium difficile, and Pseudomonas aeruginosa.

In some embodiments, the host bacteria is a drug-resistant bacteria, such as an antibiotic-resistant bacteria, and especially a multidrug-resistant (MDR) bacteria or superbug, such as methicillin-resistant Staphylococcus aureus (MRSA).

In some embodiments, the host bacteria is a bacteria having a characteristic of the ability to form a biofilm.

In some particular embodiments, the host bacteria is an S. aureus bacteria, or may otherwise be of a species such as Acinetobacter baumannii (which can cause life-threatening infections in the blood, lungs, urinary tract and in wounds), Clostridium difficile (which can cause life-threatening infections and damage to the intestinal mucosa), Pseudomonas aeruginosa (which is a common cause of chronic airway infections in cystic fibrosis patients, and which can cause serious infections in the ear, skin, blood and parts of the body following surgery), and Enterobacteriaceae species (such as E. coli, E. faecium such as vancomycin-resistant E. faecium, and Klebsiella spp, such as K. pneumoniae).

In some further particular embodiments, the host bacteria is selected from the group consisting of multidrug resistant (MDR) strains of bacteria. In addition to MDR strains of S. aureus, examples of MDR strains of A. baumannii, E. coli, Klebsiella spp, and P. aeruginosa are also particularly well known and widespread.

The host bacteria used in step (i) may, preferably, be a clinical isolate and, in particular, a clinical isolate from a subject requiring treatment for a bacterial infection; that is, the host bacteria is a sample of the bacteria causative of an infection being suffered by a subject. In some embodiments, the host bacteria is a clinical isolate from a bacterial infection characterised by the presence of a bacterial biofilm.

In some embodiments, the bacteria are gram-negative bacteria, while in some other embodiments, the bacteria are gram-positive bacteria.

Some particular examples of suitable host bacteria that may be used in step (i) include bacteria from the following species: Bacillus (eg Bacillus anthracis), Brochothrix, Corynebacterium, Enterococcus, Fingoldia (eg Fingoldia magna), Francisella (eg Francisella tularensis) Geobacillus, Gordonia, Lactobacillus, Leuconostoc, Lactococcus, Legionella, Listeria, Microcystis, Mycobacterium (eg Mycobacterium abscessus, Mycobacterium tuberculosis), Nocardia, Pediococcus, Propionibacterium, Rhodococcus, Staphylococcus (eg Staphylococcus aureus; Staphylococcus epidermidis, Staphylococcus lugdunensis) Streptococcus, Aeromonas, Azospirillum, Aggregatibacter, Bacteroides, Burkholderia, Brucella, Campylobacter, Candidatus, Caulobacter, Clavibacter, Cronobacter, Delftia, Escherichia, Erwinia, Flavobacterium, Haemophilus, Halomonas, Iodobacteria, Klebsiella, Kluyvera, Mannheimia, Morganella, Pantoea, Alphaproteobacteria, Planktothrix, Pseudoalteromonas, Pseudomonas (eg Pseudomonas aeruginosa), Pasteurella, Salmonella, Shigella, Sinorhizobium, Sodalis, Synechococcus, Thalassomonas, Thermus, Vibrio, Xanthomonas, Xylella and Yersinia. Other bacterial species are, however, also contemplated. Phage capable of infecting the abovementioned bacteria are known to those skilled in the art.

In some embodiments, the bacteria are selected from bacteria of the following genus:

Genus

Acinetobacter
Aeromonas
Agrobacterium
Arthrobacter
Azobacteroides
Bacillus
Brevibacillus
Citrobacter
Cronobacter
Enterobacter
Enterococcus
Erwinia
Escherichia
Gordonia
Klebsiella
Lactobacillus
Lactococcus
Listeria
Microbacterium
Mycobacterium
Polaribacter
Pseudoalteromonas
Pseudomonas
Ralstonia
Raoultella
Rhodococcus
Salmonella
Serratia
Shewanella
Shigella
Sinorhizobium
Staphylococcus
Streptomyces
Synechococcus
Thermoanaerobacterium
Vibrio
Yersinia

The host bacteria is treated in step (i) with a suitable amount of a phage that would normally be regarded as infectious for the bacteria if not for the circumstance that the particular bacterial strain concerned has become insensitive to the phage. For example, where the host bacteria is S. aureus, the phage may be a phage that is normally infectious for S. aureus.

Examples of Staphylococcus phage include phage from the Myoviridae, Siphoviridae, Podoviridae, Picovirinae, Spounavirinae, and Twortlikevirus families.

Examples of Staphylococcus phage include Staphylococcus phage SA1, Staphylococcus phage phi13, Staphylococcus phage phi2958PVL, Staphylococcus phage phiETA2, Staphylococcus phage phiETA3, Staphylococcus phage phiMR11, Staphylococcus phage phiMR25, Staphylococcus phage phiNM, Staphylococcus phage phiNM3, Staphylococcus phage phiPVL108, Staphylococcus phage phiPVL-CN125, Staphylococcus phage phiSauS-IPLA35, Staphylococcus phage phiSauS-IPLA88, Staphylococcus phage phiSLT, Staphylococcus phage PVL, Staphylococcus phage SAP-2, Staphylococcus phage SAP-26, Staphylococcus phage tp310, Staphylococcus phage tp310-2, Staphylococcus phage tp310-3, Staphylococcus phage Twort, Staphylococcus phage phill, Staphylococcus phage 187, Staphylococcus phage 2638A, Staphylococcus phage 37, Staphylococcus phage 3A, Staphylococcus phage 42E, Staphylococcus phage 44AHJD, Staphylococcus phage 29, Staphylococcus phage 52A, Staphylococcus phage 53, Staphylococcus phage 69, Staphylococcus phage 71, Staphylococcus phage 85, Staphylococcus phage 88, Staphylococcus phage 92, Staphylococcus phage 96, Staphylococcus phage X2, Staphylococcus phage 55, Staphylococcus phage 66, Staphylococcus phage 77, Staphylococcus phage 80alpha, Staphylococcus phage CNPH82, Staphylococcus phage K, Staphylococcus phage P954, Staphylococcus phage PH15, Staphylococcus phage phi 12, Staphylococcus phage EW, Staphylococcus phage TEM126, Staphylococcus phage S13′, Staphylococcus prophage phiPV83, Staphylococcus phage phiETA, Staphylococcus phage S24-1, and Staphylococcus phage G1.

Where the host bacteria is an Enterococcus bacterial species, then the phage used in step (i) may be selected from examples of Enterococcus phage such as those from the Siphoviridae and Myoviridae families.

Particular examples of Enterococcus phage include Enterococcus phage EFAP-1, Enterococcus phage phiEf11, Enterococcus phage phiEF24C, Enterococcus phage phiFL1A, Enterococcus phage phiFL2A, Enterococcus phage phiFL3A, Enterococcus phage phiFL4A, and Enterococcus phage SAP6.

Where the host bacteria is an Corynebacterium, then the phage used in step (i) may be selected from examples of Corynebacterium phage such as those of the Siphoviridae family.

Particular examples of Corynebacterium phage include Corynebacterium phage BFK20 and Corynebacterium phage P1201.

Where the host bacteria is a Pseudomonas, then the phage used in step (i) may be selected from examples of Pseudomonas phage including phage of the Podoviridae, Autographivirinae, Siphoviridae, Podoviridae, Myoviridae and Cystoviridae families.

Particular examples of Pseudomonas phage include Pseudomonas phage H105/1, Pseudomonas phage LKA1, Pseudomonas phage B3, Pseudomonas phage D3, Pseudomonas phage F10, Pseudomonas phage F116, Pseudomonas phage F8, Pseudomonas phage gh-1, Pseudomonas phage LBL3, Pseudomonas phage LKD16, Pseudomonas phage LMA2, Pseudomonas phage LUZ19, Pseudomonas phage PA11, Pseudomonas phage PAJU2, Pseudomonas phage PaP3, Pseudomonas phage PB1, Pseudomonas phage phi13 (S-segment), Pseudomonas phage phi15, Pseudomonas phage phi-2, Pseudomonas phage phi2954 (S-segment), Pseudomonas phage philBB-PF7A, Pseudomonas phage phikF77, Pseudomonas phage phiKZ, Pseudomonas phage PT2, Pseudomonas phage PT5, Pseudomonas phage SN, Pseudomonas phage phi297, Pseudomonas phage Bf7, Pseudomonas phage EL, Pseudomonas phageOBP, Pseudomonas phage PaP1, Pseudomonas phage 201phi21, Pseudomonas LUZ24, Pseudomonas phage phi-6 segment S, Pseudomonas phage phi8 segment S, Pseudomonas phage vB_PaeS_PMG1, and Enterobacteria phage phiKMV.

Where the host bacteria is a Bacteroides, then the phage used in step (i) may be selected from examples of Bacteroides phage include phage of the Siphoviridae family such as Bacteroides phage B40-8 and Bacteroides phage B124-14.

Where the host bacteria is a Propionibacterium, then the phage used in step (i) may be selected from examples of Propionibacterium phage include phage of the Siphoviridae family such as Propionibacterium phage PA6, Propionibacterium phage PAD20, and Propionibacterium phage PAS50.

Where the host bacteria is a Mycobacterium, then the phage used in step (i) may be selected from examples of Mycobacterium include phage of the Siphoviridae and Myoviridae families. Particular examples of Mycobacterium phage include Mycobacterium phage Muddy, Mycobacterium phage Baee, Mycobacterium phage Girr, Mycobacterium phage Hamulus, Mycobacterium phage Mozy, Mycobacterium phage Omega, Mycobacterium phage Ramsey, Mycobacterium phage Soul22, and Mycobacterium phage St Annes.

Where the host bacteria is a Yersinia, then the phage used in step (i) may be selected from examples of Yersinia such as those of the Myoviridae family. Particular examples of Yersinia phage include Yersinia phage phiR1-37.

Where the host bacteria is a Gordonia, then the phage used in step (i) may be selected from examples of Gordonia such as those of the Siphoviridae family. Particular examples of Gordonia phage include Gordonia phage Smoothie.

Further examples of suitable specific phage for use in step (i), and their respective bacterial “pair” (ie host bacteria) are shown in Table 1 below.

TABLE 1

| Bacteriophage | Host genus |
|---|---|
| Acinetobacter phage AbP2 | Acinetobacter |
| Acinetobacter phage LZ35 | Acinetobacter |
| Acinetobacter phage vB_AbaM_B09_Aci02-2 | Acinetobacter |
| Acinetobacter phage vB_AbaM_B09_Aci05 | Acinetobacter |
| Acinetobacter phage WCHABP1 | Acinetobacter |
| Acinetobacter phage YMC-13-01-C62 | Acinetobacter |
| Acinetobacter phage YMC11/12/R2315 | Acinetobacter |
| Aeromonas phage 2L372D | Aeromonas |
| Aeromonas phage 4L372D | Aeromonas |
| Aeromonas phage 4L372XY | Aeromonas |
| Aeromonas phage LAh_7 | Aeromonas |
| Agrobacterium phage Atu_ph07 | Agrobacterium |
| Arthrobacter phage CaptnMurica | Arthrobacter |
| Arthrobacter phage Circum | Arthrobacter |
| Arthrobacter phage Gordon | Arthrobacter |
| Arthrobacter phage Martha | Arthrobacter |
| Arthrobacter phage Mudcat | Arthrobacter |
| Arthrobacter phage Shade | Arthrobacter |
| Arthrobacter phage Sonny | Arthrobacter |
| Arthrobacter phage Zartrosa | Arthrobacter |
| Azobacteroides phage ProJPt-Bp1 | Azobacteroides |
| Bacillus phage AR9 | Bacillus |
| Bacillus phage BCP8-2 | Bacillus |
| Bacillus phage PBS1 | Bacillus |
| Bacillus phage SP-15 | Bacillus |
| Bacillus phage Staley | Bacillus |
| Brevibacillus phage Sundance | Brevibacillus |
| Citrobacter phage CF1 DK-2017 | Citrobacter |
| Citrobacter phage CR44b | Citrobacter |
| Cronobacter phage vB_CsaM_GAP161 | Cronobacter |
| Cronobacter phage vB_CsaP_GAP52 | Cronobacter |
| Detrevirus PMG1 | Pseudomonas |
| Enterobacter phage phiKDA1 | Enterobacter |
| Enterobacteria phage SfV | Enterobacteria |
| Enterococcus phage Ec-ZZ2 | Enterococcus |
| Enterococcus phage Idefix | Enterococcus |
| Enterococcus phage LY0322 | Enterococcus |
| Enterococcus phage vB_Efae230P-4 | Enterococcus |
| Enterococcus phage vB_EfaP_Ef6.2 | Enterococcus |
| Enterococcus phage vB_EfaP_Ef6.3 | Enterococcus |
| Enterococcus phage vB_EfaP_Ef7.2 | Enterococcus |
| Enterococcus phage vB_EfaP_Ef7.3 | Enterococcus |
| Enterococcus phage vB_EfaP_Ef7.4 | Enterococcus |
| Enterococcus phage vB_EfaP_Efmus1 | Enterococcus |
| Enterococcus phage vB_EfaP_Efmus3 | Enterococcus |
| Enterococcus phage vB_EfaP_Efmus4 | Enterococcus |
| Enterococcus phage vB_EfaP_IME195 | Enterococcus |
| Enterococcus phage vB_EfaP_IME199 | Enterococcus |
| Enterococcus phage vB_EfaP_Zip | Enterococcus |
| Enterococcus phage vB_EfaS_AL2 | Enterococcus |
| Enterococcus phage vB_EfaS_AL3 | Enterococcus |
| Enterococcus phage vB_EfaS_IME196 | Enterococcus |
| Erwinia phage PEp14 | Erwinia |
| Escherichia phage aaroes | Escherichia |
| Escherichia phage APECc02 | Escherichia |
| Escherichia phage FFH2 | Escherichia |
| Escherichia phage FV3 | Escherichia |
| Escherichia phage FV3 | Escherichia |
| Escherichia phage Henu8 | Escherichia |
| Escherichia phage HK629 | Escherichia |
| Escherichia phage JES2013 | Escherichia |
| Escherichia phage JSE | Escherichia |
| Escherichia phage LL5 | Escherichia |
| Escherichia phage Murica | Escherichia |
| Escherichia phage phT4A | Escherichia |
| Escherichia phage saus 132 | Escherichia |
| Escherichia phage slur01 | Escherichia |
| Escherichia phage slur16 | Escherichia |
| Escherichia phage SP15 | Escherichia |
| Escherichia phage Tls | Escherichia |
| Escherichia phage tonnikala | Escherichia |
| Escherichia phage V18 | Escherichia |
| Escherichia phage V18 | Escherichia |
| Escherichia phage V5 | Escherichia |
| Escherichia phage vB_EcoP_ACG-C91 | Escherichia |
| Escherichia phage vB_EcoS_HdH2 | Escherichia |
| Escherichia virus vB_Eco_mar003J3 | Escherichia |
| Fromanvirus L5 | Mycobacterium |
| Gordonia phage Avazak | Gordonia |
| Gordonia phage BrutonGaster | Gordonia |
| Gordonia phage Chikenjars | Gordonia |
| Gordonia phage Crocheter | Gordonia |
| Gordonia phage Duffington | Gordonia |
| Gordonia phage GAL1 | Gordonia |
| Gordonia phage Kenosha | Gordonia |
| Gordonia phage Neville | Gordonia |
| Gordonia phage OhMyWard | Gordonia |
| Gordonia phage Pleakley | Gordonia |
| Gordonia phage Rickmore | Gordonia |
| Gordonia phage Secretariat | Gordonia |
| Gordonia phage Smoothie | Gordonia |
| Gordonia phage Tanis | Gordonia |
| Gordonia phage Trax | Gordonia |
| Gordonia phage Untouchable | Gordonia |
| Gordonia phage Verity | Gordonia |
| Gordonia phage Yvonnetastic | Gordonia |
| Gordonia phage Zipp | Gordonia |
| Gordonia Phage Zitch | Gordonia |
| Klebsiella phage KNP2 | Klebsiella |
| Klebsiella phage KP15 | Klebsiella |
| Klebsiella phage KP27 | Klebsiella |
| Klebsiella phage KpV71 | Klebsiella |
| Klebsiella phage Matisse | Klebsiella |
| Klebsiella phage MezzoGao | Klebsiella |
| Klebsiella phage Miro | Klebsiella |
| Klebsiella phage NTUH-K2044-K1-1 | Klebsiella |
| Klebsiella phage vB_KpnM_BIS47 | Klebsiella |
| Kostyavirus eureka | Mycobacterium |
| Kryptosalinivirus M8CC19 | Bacteriodes |
| Kryptosalinivirus M8CRM1 | Bacteriodes |
| Lactobacillus phage Ldl1 | Lactobacillus |
| Lactobacillus phage phiJL-1 | Lactobacillus |
| Lactobacillus phage ViSo-2018a | Lactobacillus |
| Lactococcus lactis phage p272 | Lactococcus |
| Lactococcus phage 51701 | Lactococcus |
| Lactococcus phage 712 | Lactococcus |
| Lactococcus phage 936 | Lactococcus |
| Lactococcus phage 936 group phage Phi4.2 | Lactococcus |
| Lactococcus phage 936 group phage Phi44 | Lactococcus |
| Lactococcus phage bIL 170 | Lactococcus |
| Lactococcus phage CHPC781 | Lactococcus |
| Lactococcus phage CHPC959 | Lactococcus |
| Lactococcus phage jm3 | Lactococcus |
| Lactococcus phage P1048 | Lactococcus |
| Lactococcus phage phi15 | Lactococcus |
| Lactococcus phage PLgW-1 | Lactococcus |
| Lactococcus phage SL4 | Lactococcus |
| Lactococcus phage vB_Llc_bIBB5g1 | Lactococcus |
| Listeria phage P35 | Listeria |
| Microbacterium phage OneinaGillian | Microbacterium |
| Mycobacterium phage Acme | Mycobacterium |
| Mycobacterium phage Ariel | Mycobacterium |
| Mycobacterium phage Astraea | Mycobacterium |
| Mycobacterium phage Baee | Mycobacterium |
| Mycobacterium phage BillKnuckles | Mycobacterium |
| Mycobacterium phage Bruns | Mycobacterium |
| Mycobacterium phage Cane17 | Mycobacterium |
| Mycobacterium phage CASbig | Mycobacterium |
| Mycobacterium phage Chy4 | Mycobacterium |
| Mycobacterium phage Chy5 | Mycobacterium |
| Mycobacterium phage Courthouse | Mycobacterium |
| Mycobacterium phage D29 | Mycobacterium |
| Mycobacterium phage DD5 | Mycobacterium |
| Mycobacterium phage Edtherson | Mycobacterium |
| Mycobacterium phage Fajezeel | Mycobacterium |
| Mycobacterium phage GageAP | Mycobacterium |
| Mycobacterium phage Girr | Mycobacterium |
| Mycobacterium phage Gizmo | Mycobacterium |
| Mycobacterium phage Goku | Mycobacterium |
| Mycobacterium phage Graduation | Mycobacterium |
| Mycobacterium phage Hamulus | Mycobacterium |
| Mycobacterium phage Kingsley | Mycobacterium |
| Mycobacterium phage KristaRAM | Mycobacterium |
| Mycobacterium phage MiaZeal | Mycobacterium |
| Mycobacterium phage MosMoris | Mycobacterium |
| Mycobacterium phage Mozy | Mycobacterium |
| Mycobacterium phage Museum | Mycobacterium |
| Mycobacterium phage Nerujay | Mycobacterium |
| Mycobacterium phage Niza | Mycobacterium |
| Mycobacterium phage Omega | Mycobacterium |
| Mycobacterium phage Ovechkin | Mycobacterium |
| Mycobacterium phage Papez | Mycobacterium |
| Mycobacterium phage PattyP | Mycobacterium |
| Mycobacterium phage Pippin | Mycobacterium |
| Mycobacterium phage Quesadilla | Mycobacterium |
| Mycobacterium phage Ramsey | Mycobacterium |
| Mycobacterium phage Rufus | Mycobacterium |
| Mycobacterium phage Rutherferd | Mycobacterium |
| Mycobacterium phage Saguaro | Mycobacterium |
| Mycobacterium phage Serenity | Mycobacterium |
| Mycobacterium phage Sibs6 | Mycobacterium |
| Mycobacterium phage Solon | Mycobacterium |
| Mycobacterium phage Soul22 | Mycobacterium |
| Mycobacterium phage SpikeBT | Mycobacterium |
| Mycobacterium phage SpikeBT | Mycobacterium |
| Mycobacterium phage StAnnes | Mycobacterium |
| Mycobacterium phage SWU1 | Mycobacterium |
| Mycobacterium phage TheloniousMonk | Mycobacterium |
| Mycobacterium phage TheloniousMonk | Mycobacterium |
| Polaribacter phage P12002L | Polaribacter |
| Pseudoalteromonas phage H101 | Pseudoalteromonas |
| Pseudoalteromonas phage J2-1 | Pseudoalteromonas |
| Pseudoalteromonas phage J2-1 | Pseudoalteromonas |
| Pseudomonas phage D3 | Pseudomonas |
| Pseudomonas phage Lana | Pseudomonas |
| Pseudomonas phage PA11 | Pseudomonas |
| Pseudomonas phage PaBG | Pseudomonas |
| Pseudomonas phage phi2 | Pseudomonas |
| Pseudomonas phage phi297 | Pseudomonas |
| Pseudomonas virus Yua | Pseudomonas |
| Ralstonia phage phiRSL1 | Ralstonia |
| Ralstonia phage Raharianne | Ralstonia |
| Raoultella phage Rol | Raoultella |
| Rhodococcus phage CosmicSans | Rhodococcus |
| Rhodococcus phage CosmicSans | Rhodococcus |
| Rhodococcus phage Hiro | Rhodococcus |
| Rhodococcus phage ReqiPoco6 | Rhodococcus |
| Rhodococcus phage RER2 | Rhodococcus |
| Rhodococcus phage RGL3 | Rhodococcus |
| Rhodococcus phage Takoda | Rhodococcus |
| Rhodococcus phage Weasels2 | Rhodococcus |
| Rhodococcus phage Weasels2 | Rhodococcus |
| Salmonella phage 1-23 | Salmonella |
| Salmonella phage 103203_sal5 | Salmonella |
| Salmonella phage 2-3 | Salmonella |
| Salmonella phage 2-3 | Salmonella |
| Salmonella phage atrejo | Salmonella |
| Salmonella phage atrejo | Salmonella |
| Salmonella phage bastian | Salmonella |
| Salmonella phage fuchur | Salmonella |
| Salmonella phage LPST10 | Salmonella |
| Salmonella phage NR01 | Salmonella |
| Salmonella phage NR01 | Salmonella |
| Salmonella phage oselot | Salmonella |
| Salmonella phage rokbiter | Salmonella |
| Salmonella phage S113 | Salmonella |
| Salmonella phage S113 | Salmonella |
| Salmonella phage S126 | Salmonella |
| Salmonella phage S132 | Salmonella |
| Salmonella phage S147 | Salmonella |
| Salmonella phage SE24 | Salmonella |
| Salmonella phage Seszw_1 | Salmonella |
| Salmonella phage SETP13 | Salmonella |
| Salmonella phage SETP7 | Salmonella |
| Salmonella phage SH9 | Salmonella |
| Salmonella phage Shivani | Salmonella |
| Salmonella phage Shivani | Salmonella |
| Salmonella phage SP01 | Salmonella |
| Salmonella phage STG2 | Salmonella |
| Salmonella phage vB_SenS_AG11 | Salmonella |
| Salmonella phage vB_SenS_SB28 | Salmonella |
| Serratia phage PS2 | Serratia |
| Shewanella sp. phage 1/4 | Shewanella |
| Shewanella sp. phage 1/40 | Shewanella |
| Shigella phage SFN6B | Shigella |
| Shigella phage SHSML-45 | Shigella |
| Shigella phage SHSML-45 | Shigella |
| Sinorhizobium phage phiM7 | Sinorhizobium |
| Sinorhizobium phage phiN3 | Sinorhizobium |
| Staphylococcus phage 676Z | Staphylococcus |
| Staphylococcus phage 676Z | Staphylococcus |
| Staphylococcus phage 812 | Staphylococcus |
| Staphylococcus phage 812 | Staphylococcus |
| Staphylococcus phage 85 | Staphylococcus |
| Staphylococcus phage A3R | Staphylococcus |
| Staphylococcus phage A3R | Staphylococcus |
| Staphylococcus phage A5W | Staphylococcus |
| Staphylococcus phage A5W | Staphylococcus |
| Staphylococcus phage Fi200W | Staphylococcus |
| Staphylococcus phage Fi200W | Staphylococcus |
| Staphylococcus phage G1 | Staphylococcus |
| Staphylococcus phage G1 | Staphylococcus |
| Staphylococcus phage IME-SA1 | Staphylococcus |
| Staphylococcus phage IME-SA1 | Staphylococcus |
| Staphylococcus phage IME-SA118 | Staphylococcus |
| Staphylococcus phage IME-SA118 | Staphylococcus |
| Staphylococcus phage IME-SA119 | Staphylococcus |
| Staphylococcus phage IME-SA119 | Staphylococcus |
| Staphylococcus phage IME-SA2 | Staphylococcus |
| Staphylococcus phage IME-SA2 | Staphylococcus |
| Staphylococcus phage ISP | Staphylococcus |
| Staphylococcus phage ISP | Staphylococcus |
| Staphylococcus phage K | Staphylococcus |
| Staphylococcus phage K | Staphylococcus |
| Staphylococcus phage MCE-2014 | Staphylococcus |
| Staphylococcus phage MSA6 | Staphylococcus |
| Staphylococcus phage MSA6 | Staphylococcus |
| Staphylococcus phage P4W | Staphylococcus |
| Staphylococcus phage P4W | Staphylococcus |
| Staphylococcus phage philBB-SEP1 | Staphylococcus |
| Staphylococcus phage phiIPLA-RODI | Staphylococcus |
| Staphylococcus phage S25-3 | Staphylococcus |
| Staphylococcus phage S25-4 | Staphylococcus |
| Staphylococcus phage SA5 | Staphylococcus |
| Staphylococcus phage SA5 | Staphylococcus |
| Staphylococcus phage Sb1 | Staphylococcus |
| Staphylococcus phage Sb1 | Staphylococcus |
| Staphylococcus phage Staph1N | Staphylococcus |
| Staphylococcus phage Staph1N | Staphylococcus |
| Staphylococcus phage StB20 | Staphylococcus |
| Staphylococcus phage Team1 | Staphylococcus |
| Staphylococcus phage Team1 | Staphylococcus |
| Staphylococcus phage Twort | Staphylococcus |
| Staphylococcus phage X2 | Staphylococcus |
| Streptomyces phage Bing | Streptomyces |
| Streptomyces phage Braelyn | Streptomyces |
| Streptomyces phage DrGrey | Streptomyces |
| Streptomyces phage Gilson | Streptomyces |
| Streptomyces phage Paradiddles | Streptomyces |
| Streptomyces phage Rima | Streptomyces |
| Streptomyces phage Samisti12 | Streptomyces |
| Streptomyces phage Scap1 | Streptomyces |
| Streptomyces phage Wollford | Streptomyces |
| Synechococcus phage S-CBS3 | Synechococcus |
| Synechococcus phage S-CBS4 | Synechococcus |
| Thermoanaerobacterium phage THSA-485A | Thermoanaerobacterium |
| Tlsvirus SP126 | Escherichia |
| Vibrio phage 11895-B1 | Vibrio |
| Vibrio phage 11895-B1 | Vibrio |
| Vibrio phage eugene 12A10 | Vibrio |
| Vibrio phage helene 12B3 | Vibrio |
| Vibrio phage pYD21-A | Vibrio |
| Vibrio phage qdvp001 | Vibrio |
| Vibrio phage VP93 | Vibrio |
| Vibrio virus vB_VspP_SBP1 | Vibrio |
| Vibrio virus vB_VspP_SBP1 | Vibrio |
| Yersinia phage phiR1-37 | Yersinia |
| Yersinia phage phiR1-37 | Yersinia |

The phage may be selected from available stocks of phage such as may be found in a “phage bank”, or identified by screening natural sources of phage. The phage used to treat the host bacteria are referred to as the “entry phage”. Those skilled in the art will be able to routinely identify a suitable amount of phage for the treatment of the host bacteria, but typically, the phage will be provided in an amount corresponding to a multiplicity of infection (MOI) value in the range of 0.05 to 2.0, more preferably about 0.5 to 1.2, and most preferably, of about 1.0. In this regard, the MOI may be 1.0 or more, 0.5 or more, 0.1 or more, or 0.001 or more, or less than 1.0, less than 0.1, or less than 0.01.

The antibacterial agent used to treat the host bacteria in step (i) comprises protein synthesis inhibitor activity. Protein synthesis inhibitors (PSIs) may act via a mechanism of an interruption of peptide chain elongation, the blocking of the decoding site (A site) of ribosomes, the misreading of the genetic code or the prevention of the attachment of oligosaccharide side chains to glycoproteins. Antibacterial agents such as PSI antibiotics are well known to those skilled in the art.

In some embodiments, the antibacterial agent comprising protein synthesis inhibitory activity comprises an agent which binds to the 50S ribosomal subunit and inhibits protein synthesis by preventing peptide chain elongation. Examples include macrolides such as clindamycin, erythromycin, clarithromycin and azithromycin.

In some embodiments, the antibacterial agent comprising protein synthesis inhibitory activity comprises an agent that can inhibit protein synthesis by blocking the A site of the 30S ribosomal subunit. Examples include kanamycin and streptomycin.

Examples of protein synthesis inhibitors suitable for use in step (i) include spectinomycin, tetracycline, clindamycin, azithromycin, kanamycin, gentamicin, netilmicin, linezolid, chloramphenicol, capreomycin, mupirocin, dactinomycin, telithromycin, neomycin, lincomycin, fusidic acid, cethromycin, viomycin, sisomycin, puromycin and streptomycin.

In some embodiments, the host bacteria is treated in step (i) with an amount of the antibacterial agent that is equal to or greater than the MIC, for example 2×MIC, or 1×MIC.

In some embodiments, the host bacteria is treated in step (i) with a sub-inhibitory amount of at least one antibacterial agent. Those skilled in the art will be able to routinely identify a sub-inhibitory amount of the antibacterial agent for the treatment of the host bacteria, but typically, where the antibacterial agent is an antibiotic such as an antibiotic to which the host bacteria is sensitive, the sub-inhibitory amount will be in the range of about 0.125 to 0.95×MIC (minimum inhibitory concentration), more preferably about 0.125 to 0.65×MIC and most preferably, about 0.5×MIC (“½ MIC”). Other concentrations are, however, also contemplated.

In some embodiments, the present disclosure provides a method of producing a modified phage, said method comprising the steps of:
(i) treating a host bacteria insensitive to a selected phage strain (ie a phage non-sensitive, or phage insensitive, or phage resistant bacteria) with a suitable amount of the said phage strain and a sub-inhibitory amount of at least one antibacterial agent comprising protein synthesis inhibitor activity, and culturing the bacteria for a period and under conditions suitable for the phage to infect the bacteria and replicate to generate progeny phage; and
(ii) recovering and expanding progeny phage which are phage with a modified capability to infect and cause lysis of said host bacteria.

The treatment with the antibacterial agent may be performed concurrently with the phage treatment, before treatment with the phage, and/or after treatment with the phage.

Typically, the treatment with the antibacterial agent is performed before the treatment with the phage (ie before the phage is added) in what is effectively a pre-treatment with the antibacterial agent. The pre-treatment with the antibacterial agent may be commenced up to 10 hours, more preferably up to 6 hours, more preferably up to 4 hours, more preferably up to 3 hours, prior to contacting the host bacteria with the entry phage. However, in some embodiments, the phage may be added first, then followed by the addition of the antibacterial agent.

The host bacteria treated in accordance with step (i) are cultured for a period and under conditions suitable for the phage to infect the bacteria and replicate to generate progeny phage. Those skilled in the art are able to readily determine such a period and conditions. However, typically, the culture will be performed on standard growth media for 24 hours at 37° C. The progeny phage are also referred to as the “exit phage”. Exit phage may be readily purified from the culture of step (i), if desired, by standard methodologies known to those skilled in the art including, for example, a purification process comprising centrifugation of the culture (to remove residual bacteria and bacterial cell walls etc. resulting from lysis) and filtration (eg by using a 0.2 μm syringe filter) to purify the phage particles. The purified exit phage may be used in step (ii) of the method.

In step (ii) of the method of the first aspect, exit phage (progeny phage) that have a modified capability to infect and cause lysis and killing of the host bacteria (eg exit phage which show a modified, enhanced, level of bactericidal activity against the host bacteria or “parent” used in step (i) relative to the entry phage) are recovered and propagated to expand the number of phage (ie the number of phage particles is increased) for subsequent use in, for example, a method of treating a subject requiring treatment for a bacterial infection or for storage in, for example, a phage bank. This may involve one or more steps (preferably, two or more steps) of culturing the host bacteria in the presence of the exit phage, or in other words, one or more steps of passaging the exit phage through the host bacteria. These culturing/passaging steps preferably do not include any treatment with the antibacterial agent as used in step (i) of the method, but may do so. Otherwise, the exit phage may be propagated by culturing/passaging with other sensitive bacteria to expand the number of phage. Phage particles that are present after these culturing/passaging steps represent modified exit phage that are capable of infecting and causing lysis of the host bacteria. Optionally, these exit phage may undergo one or more selection or analysis steps including, but not limited to: identifying exit phage with markedly enhanced lytic activity (eg to identify modified phage capable of infecting the parent bacteria only, but with at least a 500-fold or, more preferably, 1000-fold higher level of lytic activity; identifying exit phage with stability and/or growth (replication) characteristics that are substantially unchanged from that of the entry phage, except for a higher burst size; and/or identifying exit phage which encode and produce a lysin protein with markedly enhanced lytic activity which may involve, for example, assessing the relative lytic activity of the lysin protein produced by the modified phage (eg by expressing an isolated nucleic acid molecule encoding the lysin protein in a host bacteria: see for example the PHEARLESS assay described in the examples hereinafter) or through the sequencing of at least a lysin-encoding nucleotide sequence within the exit phage genome. It will be readily appreciated by those skilled in the art that the method of the first aspect may be performed in a multiple (parallel) format where different entry phage are separately used to treat different samples of host bacteria to generate different progeny exit phage; and the use of the optional one or more selection or analysis steps may then be used to identify a preferred exit phage from amongst the separately generated exit phage candidates.

In some embodiments, the exit phage may be further propagated using the host bacteria or other sensitive phage to further expand the number of phage, such as for the purpose of storage in a phage bank.

The method of the first aspect may be advantageous inasmuch as the use of repeated steps for the generation of modified phage may be avoided. Thus, step (i) preferably consists of no more than three steps, or no more than two steps, and more preferably only a single step of treating the insensitive host bacteria with a suitable amount of the selected phage strain and at least one antibacterial agent, and culturing the bacteria for a period and under conditions suitable for the phage to infect the bacteria and replicate to generate progeny phage. Moreover, in step (ii), where the exit phage is expanded (as described in paragraph [0068]), the steps of culturing/passaging are preferably not conducted using the host bacteria in combination with at least the antibacterial agent that was used in step (i) or, more preferably, any other antibacterial agent comprising protein synthesis inhibitor activity.

As such, the method of the first aspect may produce a modified exit phage to which the host bacteria has been re-sensitised, and which may be of use in (for example) treating a subject requiring treatment for a bacterial infection (ie in a phage therapy). Thus, in some embodiments, the method of the first aspect may be used to produce a modified exit phage which, unlike the selected entry phage, is capable of infecting and causing lysis of a host bacteria (such as a clinical isolate) causative of an infection being suffered by a subject.

Thus, in some embodiments, the method of the first aspect is for producing a modified phage for use in a phage therapy of a subject's infection, and comprises the steps of:
(i) treating a host bacteria that has been isolated from the said subject and which is insensitive to a selected phage strain (ie a phage non-sensitive bacteria) with a suitable amount of the said phage strain and at least one antibacterial agent comprising protein synthesis inhibitory activity, and culturing the bacteria for a period and under conditions suitable for the phage to infect the bacteria and replicate to generate progeny phage;
(ii) recovering and expanding progeny phage which are phage with a modified capability to infect and cause lysis of said host bacteria; and
(iii) providing the modified phage in a pharmaceutical composition for administration to the said subject.

In some embodiments, the amount of the antibacterial agent comprising protein synthesis inhibitory is activity is a sub-inhibitory amount.

Such embodiments enable a “personalised” approach to phage therapy which may be employed, for example, where a phage with an effective level of bactericidal activity against a bacteria causative of an infection in a subject, has either not been identified or is not readily available. As such, the method may enable the selection of an available phage that would normally be regarded as infectious for the bacteria if not for the circumstance that the particular bacterial strain concerned has become insensitive to the phage, and to use that phage as an entry phage from which to produce a modified exit phage that may then enable an effective phage therapy of the infection in the subject.

Thus, in a second aspect, the present disclosure provides a method of treating a bacterial infection in a subject, said method comprising the steps of:
1. obtaining a sample of a bacteria causative of an infection in said subject,
2. treating the bacteria with a suitable amount of a selected phage strain to which the bacteria is insensitive (ie the bacteria is phage non-sensitive) and at least one antibacterial agent comprising protein-synthesis inhibitory activity, and culturing the bacteria for a period and under conditions suitable for the phage to infect the bacteria and replicate to generate progeny phage;
3. recovering and expanding progeny phage which are phage with a modified capability to infect and cause lysis of said host bacteria; and
4. administering to the subject an effective amount of the progeny phage, or a further propagated form thereof, to treat the infection.

In this second aspect, the present disclosure provides a method of treating a bacterial infection in a subject, the method comprising producing phage with improved lytic activity against a phage insensitive bacteria by exposing the insensitive host bacterium to a selected infecting phage and an agent having protein synthesis inhibitory activity and treating the infection with an effective amount of the phage with improved lytic activity.

In some embodiments, the present disclosure provides a method of treating a bacterial infection in a subject, said method comprising the steps of:
1. obtaining a sample of a bacteria causative of an infection in said subject,
2. treating the bacteria with a suitable amount of a selected phage strain to which the bacteria is insensitive (ie the bacteria is phage non-sensitive) and a sub-inhibitory amount of at least one antibacterial agent comprising protein-synthesis inhibitory activity, and culturing the bacteria for a period and under conditions suitable for the phage to infect the bacteria and replicate to generate progeny phage;
3. recovering and expanding progeny phage which are phage with a modified capability to infect and cause lysis of said host bacteria; and
4. administering to the subject an effective amount of the progeny phage, or a further propagated form thereof, to treat the infection.

The method of the second aspect may, particularly, be used to treat an infection in the subject which is not responding to antibiotic-based therapy (thereby indicating that the infectious bacteria is antibiotic drug-resistant) and/or treatment with phage (thereby indicating that the infectious bacteria is phage resistant or phage insensitive). The bacterial sample may be obtained from (eg isolated from), for example, a suitable body sample from the subject such as blood, urine, faecal sample, swab, or a tissue biopsy (eg a biopsy from a tissue at the site of infection). Bacteria present in the body sample may be isolated and expanded according to any of the standard methodologies known to those skilled in the art. The bacteria may optionally be identified by using standard methodologies including microscopy techniques, tests such as latex agglutination tests (see, for example, Essers L., J Clin Microbiol 12 (5): 641-643, 1980), MALDI and/or 16 S rRNA gene sequencing such as is well known to those skilled in the art. On the basis of the identification of the bacteria, a phage that would normally be regarded as infectious for the bacteria may be selected from, for example, a phage bank for use as the “entry phage”, however more typically, the selected phage will be a phage previously employed in a phage therapy on the subject (which was unsuccessful) or to which the causative bacteria has been found to be insensitive in, for example, a standard in vitro phage sensitivity test. The bacteria may be treated with the phage and antibacterial agent as described above in relation to the method of the first aspect. The treatment of the bacteria with the antibacterial agent is typically performed before the phage treatment. The step of recovering and expanding the progeny “exit phage” (eg to provide sufficient numbers of the modified exit phage for use in a phage therapy) may be carried out as described above in relation to the method of the first aspect. As such, the modified exit phage may also be further propagated using, for example, a sensitive bacterial strain, as required (eg for storage in a phage bank). For administration to the subject, the modified exit phage (or a further propagated form thereof) will typically be provided as a pharmaceutical composition comprising the phage in combination with at least one pharmaceutically acceptable carrier, diluent and/or excipient. The modified exit phage (or a further propagated form thereof) may be administered to the subject by any suitable route such as topical administration (eg for treatment of a surface wound), intramuscular administration (im) (eg by injection), intravenous administration (iv) (especially systemic infusion), oral administration or airway administration (eg by rinsing, nebulisation, spraying into the nose and paranasal sinuses and into the lungs in pulmonary administration). In some embodiments, the modified phage (or a further propagated form thereof) may be administered with one or more additional phage strain(s) (ie in a “phage cocktail”) which may contribute to the effectiveness of the phage therapy in treating the infection, particularly in the case of a polymicrobial infection (ie where more than one bacteria strain is present in the infection). In addition, or alternatively, the modified phage (or a further propagated form thereof) may be used in a combination therapy with a suitable antibacterial agent.

The method of the second aspect may be used to treat a bacterial infection in a subject, and especially a bacterial infection caused by bacteria showing drug-resistance and/or which is phage insensitive. In some embodiments, the method of the second aspect may be used to treat an infection in a subject caused by methicillin-resistant Staphylococcus aureus (MRSA) (eg a skin or lung infection, diabetic foot ulcer, sinus infection or sepsis), A. baumannii (eg an infection of the blood (bacteremia), lungs or urinary tract, or a wound infection by A. baumannii), C. difficile (eg a colon infection), P. aeruginosa (eg a lung infection, bacteremia, bone and joint infections, post-surgery infection, diabetic foot ulcers, sinus infection, ear infection), MDR strains of E. coli (eg an urinary tract infection (UTI)), an E. faecium such as vancomycin-resistant E. faecium (eg an infection of the blood (bacteremia), urinary tract, or of a wound), and Klebsiella spp, such as K. pneumoniae (eg an infection of the lungs or a UTI). Further, in one particular embodiment, the method of the second aspect may be used to treat sinusitis or rhinosinusitis (acute rhinosinusitis and chronic rhinosinusitis) which is commonly caused by S. aureus and/or P. aeruginosa.

In this specification, a number of terms are used which are well known to those skilled in the art. Nevertheless, for the purposes of clarity, a number of these terms are hereinafter defined.

As used herein, the term “sub-inhibitory amount” refers to an amount (concentration) of an antibacterial agent which is below that required to detectably inhibit the growth and replication of a particular bacteria. The sub-inhibitory amount is a non-lethal amount for the particular bacteria.

The term “minimum inhibitory concentration” is to be understood as it is to those skilled in the art. Therefore, as used herein, the term refers to the lowest amount (concentration) of an antibacterial agent that detectably inhibits the growth of a particular bacteria.

The terms “phage non-sensitive”, “phage insensitive” and “phage resistant” are used interchangeably to describe a characteristic of a particular bacteria to be insensitive to a certain phage or phages, such that phage(s) when contacted with the bacteria is unable to substantially infect and/or cause lysis of the bacteria (eg as may be detected by standard in vitro phage sensitivity test where lysis zones are assessed after spotting of phage onto a bacterial lawn on a standard petri dish or plate, and overnight culture). Such bacteria may be partially or fully resistant to the particular phage or phages. In contrast, phage contacted with “sensitive” bacteria will infect and/or cause lysis of the bacteria.

The term “lysis” refers to a process whereby, for example, a phage or chemical agent causes the breaking down of the wall or membrane of a cell such as a bacterial cell leading to cell death. In the context of the present disclosure, the modified exit phage may show enhanced “lytic activity”, meaning that the modified phage has an enhanced (increased) capability to cause lysis and/or killing of the host bacteria as compared to the entry phage. This lytic activity may be achieved, wholly or in part, by phage proteins known as “endolysins” (lysins) which are peptidoglycan-degrading or endopeptidase enzymes which can breakdown bacterial cell walls.

As used herein, the term “treating” includes prophylaxis as well as the alleviation of established symptoms of an infectious disease or condition. As such, the act of “treating” an infection in a subject therefore includes: (1) preventing or delaying the appearance of clinical symptoms of the infectious disease or condition developing in a subject afflicted with the disease or condition; (2) inhibiting the infectious disease or condition (ie arresting, reducing or delaying the development of a bacterial infection or a relapse thereof (in case of a maintenance treatment) or at least one clinical or subclinical symptom thereof); and (3) relieving or attenuating the infectious disease or condition (ie causing regression of the bacterial infection or at least one of its clinical or subclinical symptoms). Treatment encompasses single or multiple treatments.

As used herein, the phrase “manufacture of a medicament” includes the use of the modified phage directly as a medicament, in combination with other additives, or in any stage of the manufacture of a medicament.

The term “effective amount” refers to an amount sufficient to effect beneficial or desired clinical results (ie it is a “therapeutically effective amount”). A therapeutically effective amount can be administered in one or more administrations. Typically, a therapeutically effective amount is sufficient for treating an infectious disease or condition, or otherwise to palliate, ameliorate, stabilise, reverse, slow or delay the progression of such a disease or condition. By way of example only, a therapeutically effective amount of a modified phage produced in accordance with the method of the first aspect may comprise 10⁴ to 10¹² PFU, and more preferably 10⁸ to 10¹⁰ PFU. However, notwithstanding the above, it will be understood by those skilled in the art that the therapeutically effective amount may vary and depend upon a variety of factors including the activity of particular phage(s), the age, body weight, sex and health of the subject, the route and time of administration of the modified phage, and the severity of, for example, the infectious disease or condition, and the type or types of bacteria causative of the infection.

As mentioned above, the modified phage may be used in a combination therapy with a suitable antibacterial agent, in which case, the phage and antibacterial agent (eg an antibiotic) may be administered to the subject consecutively, together or in either order. As used herein, it is to be understood that when administered consecutively, the phage and antibacterial agent may be administered one after another with practically no time interval (ie one is administered effectively immediately after the other) or, otherwise, after an interval of 1 to 5 minutes or more (eg 10 minutes, 30 minutes, 60 minutes, 4 hours or 6 hours). Accordingly, in some embodiments, the phage and the antibacterial agent are provided in combination for administration in a single pharmaceutical composition. Such a pharmaceutical composition may further comprise, for example, a pharmaceutically acceptable carrier, diluent and/or excipient, and may be suitable for, for example, topical administration, intramuscular administration (im), oral administration or intravenous administration (iv). On the other hand, in some embodiments, where the phage and the antibacterial agent are administered consecutively, they may each be formulated for administration in a pharmaceutical composition form including a pharmaceutically acceptable carrier, diluent and/or excipient, which may be the same or different. Again, the pharmaceutical compositions may be suitable for, for example, topical administration, intramuscular administration (im), oral administration or intravenous administration (iv).

In a third aspect, the present disclosure provides a modified phage produced in accordance with the method of the first aspect. The phage may be further propagated using, for example, a sensitive bacterial strain, as required.

In a fourth aspect, the present disclosure provides a pharmaceutical composition comprising a modified phage produced in accordance with the method of the first aspect, or a further propagated from thereof, optionally in combination with at least one pharmaceutically acceptable additive, carrier, diluent and/or excipient.

Such a pharmaceutical composition may also comprise one or more additional phage strain(s) (ie in a “phage cocktail”) and/or a suitable antibacterial agent (ie for a combination therapy). In addition, the pharmaceutical composition may optionally comprise other substances such as absorption enhancers including surfactants (eg sodium lauryl sulphate, laureth-9, sodium dodecylsulphate, sodium taurodihydrofusidate and poly oxyethylene ethers), chelating agents (eg EDTA, citric acid and salicylates), and preservatives (eg sodium benzoate, sorbic acid and esters of p-hydroxybenzoic acid) and/or suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

The at least one pharmaceutically acceptable additive, carrier, diluent and/or excipient may be selected from any of the suitable additives, carriers, diluents and excipients that are well known to those skilled in the art, and which are described in, for example, Remington's Pharmaceutical Sciences, Mack Publishing Co., Easton, PA 1995. Particular examples of suitable carriers include lactose, starch, glucose, methyl cellulose, magnesium stearate, mannitol, sorbitol and the like. Examples of suitable diluents include ethanol, glycerol and water. Particular examples of suitable excipients may be found in the Handbook of Pharmaceutical Excipients, 2nd Edition, (1994), Edited by A Wade and PJ Weller.

The pharmaceutical compositions may be intended for single daily administration, multiple daily administration, or controlled or sustained release, as needed to achieve the most effective results.

Examples of pharmaceutical compositions include compositions suitable for delivery by injection, compositions suitable for intravenous administration, compositions suitable for delivery by topical administration, compositions suitable for delivery via a gel, and compositions suitable for pulmonary delivery. For example, a suitable composition for topical delivery may comprise a requisite concentration of phage in PBS, in water or in SM buffer (To prepare: NaCl, 5.8 g, 100 mM; MgSO4.7H2O 2 g, 8 mM; Tris-Cl (1 M, pH 7.5), 50 ml, 50 mM; H2O to 1 liter).

In a fifth aspect, the present disclosure provides the use of a modified phage produced in accordance with the method of the first aspect, or a further propagated form thereof, in the manufacture of a medicament for treating an infection in a subject. Methods for manufacturing medicaments are known in the art.

In a sixth aspect, the present disclosure provides a modified phage, produced in accordance with the method of the first aspect, or a further propagated form thereof, for treating an infection in a subject.

Examples of diseases or conditions suitable for treatment with a modified phage are described herein include sinus conditions (eg acute rhinosinusitis and chronic rhinosinusitis), respiratory conditions (eg lung infections, cystic fibrosis), ear conditions (infections of the external ear canal or of the middle ear), diabetic ulcers (eg diabetic foot ulcers), sepsis or a skin infection (eg Staphylococcus skin infections). Other diseases or conditions are contemplated.

The modified phage of the present disclosure is typically applied to the treatment of an infectious disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

In a seventh aspect, the present disclosure provides a method of killing bacteria, said method comprising contacting the bacteria with a modified phage produced in accordance with the method of the first aspect or a further propagated form thereof.

The method of the seventh aspect may be conducted in, for example, a laboratory setting where, for example, the method may be used to prevent bacterial contamination, and other situations where bacterial infections or contaminations can occur (eg industrial and environmental situations). In some embodiments, the method of the seventh aspect may be used for decontaminating surfaces and equipment such as food preparation surfaces and equipment, and medical and surgical surfaces and/or equipment etc. The bacteria for killing may be present in a planktonic or biofilm form.

As described herein, the method of the seventh aspect may also be used for the treatment of a subject.

As mentioned above, the modified phage produced in accordance with the method of the first aspect, are capable of infecting and causing lysis of the parent host bacteria. In some embodiments, the modified phage may show markedly enhanced lytic activity of a level which is at least 1000-fold higher than that of the entry phage. Such modified phage may express a novel lysin protein with markedly enhanced lytic activity.

Thus, in an eighth aspect, the present disclosure provides an isolated phage lysin protein (an endolysin) derived from a modified phage produced in accordance with the method of the first aspect.

The lysin protein may also be formulated into a composition, such as a pharmaceutical composition for use in treating a bacterial infection in a subject (such as those described above), or for killing bacteria in other situations such as environmental contaminations, or for decontaminating surfaces and equipment such as some of those mentioned above including food preparation surfaces and equipment, surgical equipment and air conditioning systems etc.

Standard techniques and equipment may be used for recombinant DNA technology, DNA sequencing, oligonucleotide synthesis, molecular biology, cell biology and enzymatic reactions, which may be generally performed according to methods known in the art and/or as commercially available, and are as described for example in Sambrook et al. Molecular Cloning: A Laboratory Manual (4th ed., Cold Spring Harbor Laboratory Press, Cold Spring Harbor, N.Y. (1989)) and Ausubel et al Current Protocols in Molecular Biology (2003) John Wiley & Sons, both of which are herein incorporated by reference.

The methods, phage and phage proteins (lysins) of the present disclosure are hereinafter further described with reference to the following non-limiting examples and accompanying figures.

### EXAMPLES

### Example 1

The study described in this example aimed to evaluate whether specific antibiotics could sensitise phage non-sensitive S. aureus isolates to phage infection in vitro and in vivo.

### Materials and Methods

### Phages, Clinical Isolates and Antibiotics

S. aureus ATCC51650 and ATCC25923 were obtained from the American Type Culture Collection (Manassas, VA, United States of America) and used as reference strains. In addition, a total of 15 S. aureus clinical isolates (CIs) were isolated from the sinonasal cavities of chronic sinusitis (CRS) patients. In each case, S. aureus was cultured by an independent diagnostic laboratory and the antibiotic susceptibility determined by disc diffusion on Mueller-Hinton agar per Clinical and Laboratory Standards Institute (CLSI) recommendations (Adelaide Pathology Partners, Mile End, SA, Australia). Bacteria were isolated on Columbian horse blood agar plates, colistin nalidixic acid plates, or cystine lactose electrolyte-deficient plates (Oxoid Australia Pty Ltd, Thebarton, SA, Australia). Positive cultures were confirmed to be S. aureus using latex agglutination testing. Phages J-Sa36 (Sa36), Sa83 and Sa87 were obtained from AmpliPhi Australia Pty Ltd (Brookvale, NSW, Australia).

### Antibiotic Sensitivity Testing

The minimum inhibitory concentration (MIC) of amoxicillin, vancomycin, erythromycin, clindamycin and azithromycin against the S. aureus strains was assessed using the broth microdilution technique as previously described (Wiegand I et al., Nature protocols 3 (2): 163-175, 2008) and breakpoints defined according to CSLI guidelines. Clinical strains resistant to at least one antibiotic in three or more antibiotic categories were defined as multidrug resistant (MDR).

### Phage Sensitivity Test

The sensitivity of S. aureus isolates (15 clinical isolates and 2 ATCC strains) to phages J-Sa36, Sa83 and Sa87 was determined as previously described (Drilling A et al., Am J Rhinol Allergy 28 (1): 3-11, 2014). Briefly, 0.5 McFarland Units of S. aureus CIs diluted 1:100 in 10 ml Tryptic soy broth (TSB) were grown overnight at 37° C. with shaking. 200 μL of overnight broth cultures were then spread onto Columbian blood agar plates evenly (Oxoid). Plates were incubated at 37° C. for 1 hour and 5 μL [10⁶ plaque forming units (PFU)] of 3 phages were spotted in triplicates onto the plates and incubated at 37° C. overnight. Phage buffer solution (SM buffer) alone was spotted in the centre of the plates as negative control. Phage sensitivity was determined according to the protocol described in Zhang G et al., editors. Bacteriophage effectively kills multidrug resistant Staphylococcus aureus clinical isolates from chronic rhinosinusitis patients. International Forum of Allergy & Rhinology; 2018: Wiley Online Library. Confluent lysis zones observed on plates were defined as phage sensitive, semi-confluent lysis zones were defined as phage semi-sensitive, and no plaques were defined as phage insensitive (non-sensitive).

Determining the Effect of Phage Combined with Antibiotics on Planktonic S. aureus

Initial screening was performed in 100 μl in 96 well plates, with follow up experiments in 10 mL culture volume. S. aureus strains were grown on 1.5% Tryptone Soya Agar (TSA) at 37° C. overnight. Single colonies were transferred to Tryptone Soya Broth (TSB) to generate a culture with a starting concentration of 5×10⁵ CFU/ml. Phage J-Sa36, Sa83 and Sa87 were added to the cultures at multiplicity of infection (MOI) of 0.2 according to predetermined phage titers (described below). Antibiotics were added to the cultures at ½ MIC (according to the pretest MIC of antibiotics) if required. Optical density (OD) readings at 600 nm were recorded at 24 hours to assess the growth of bacteria co-inoculated with phage unless otherwise specified. Uninoculated, fresh media was included as a sterility control.

### Determination of Phage Titers

Phage titers were determined against S. aureus RN4220 (ATCC) using the double layer spot assay technique. Briefly, 100 μl of a S. aureus overnight culture was mixed with 4 mL of 0.4% TSA and poured onto a 1.5% TSA plate. After 20 minutes, serially diluted samples of phage J-Sa36, Sa83 or Sa87 were spotted onto the double layer agar plates in 5 μl spot volumes in triplicates. After the plates were incubated in a humidified incubator at 37° C. overnight, plaques were counted, counts were averaged, and titers of the filtrate were calculated based on the dilution.

### Determining Phage Replication by Titer

A 100 μl sample was taken from an S. aureus culture (10 ml in TSB) after 0 and 48 hours following treatment with clindamycin, phage or a phage clindamycin co-incubation. The sample was treated with 25 μl of chloroform, shaken for 30 seconds, centrifuged (3000 rpm, 5 minutes, room temperature) and the supernatant used to determine phage titers against the same isolate. Phage titers in plaque forming units [PFU]/mL were recorded initially and at 48 hours to assess for phage replication. To allow for phage resistant (phage insensitive) bacterial isolates to show plaques, clindamycin was added to the soft agar overlay.

Determining the Effect of Phage in Combination with Clindamycin on S. aureus Biofilms

The effect of J-Sa36, Sa83 and Sa87 combined with clindamycin was tested on S. aureus biofilms formed by ATCC51650 and clinical isolate 11 (CI11). Briefly, a 1.0 McFarland unit S. aureus culture suspended in 1:15 dilution in TSB was added to 96-well microtiter plates (Costar; Corning Incorporated, Corning, NY, United States of America). Plates were incubated for 48 hours at 37° C. on a rotating platform at 70 rpm (3D Gyratory Mixer; Ratek Instruments, Boronia, VIC, Australia) to allow biofilm formation followed by washing with phosphate-buffered saline. Biofilms were then treated with 8×10⁶ plaque forming units (PFU) of phage J-Sa36, Sa83 or Sa87 and clindamycin added with a final concentration of 1 μg/ml, 0.5 μg/ml, 0.2 μg/ml, 0.1 μg/ml, or 0.05 μg/ml, in TSB. Wells were treated for 24 hours at 37° C. with gentle shaking. The AlamarBlue viability assay (Thermo Fisher Scientific, Waltham, MA, United States of America) was used as previously described (Richter K et al., editors. Mind “De GaPP”: in vitro efficacy of deferiprone and gallium-protoporphyrin against Staphylococcus aureus biofilms. International Forum of Allergy & Rhinology; 2016: Wiley Online Library to determine the anti-biofilm activity of the phage. Fluorescence was measured in a microplate reader (FLUOstar OPTIMA, BMG LABTECH, Offenburg, Germany) and the percent reduction of biofilm after treatment determined relative to untreated controls.

Time-Dependent Effect of the Interaction of Phage J-Sa36 and Clindamycin in S. aureus
(1) ATCC51650 or CI11 was incubated with ½ MIC clindamycin in 10 ml TSB, then phage J-Sa36 (1×10⁵ PFU/ml) was added into the above at different times (1h, 2 h, 3h, 3.5h, 4 h, 6 h and 24 h) and incubated for 24 hours. OD600 readings were taken at phage addition and following 24-hour incubation.
(2) ATCC51650 or CI11 was incubated with either ½ MIC clindamycin or phage J-Sa36 (MOI=0.2) for 3.5 hours followed by the addition of phage J-Sa36 (1×10⁵ PFU/ml, MOI=0.2) or ½ MIC clindamycin respectively and continuous incubation with both ½ MIC clindamycin and phage J-Sa36 for 24 hours. OD600 readings were taken at addition of either phage or clindamycin and following the 24-hour incubation.

The total amount of phage J-Sa36 added into the culture at different time points was adjusted to maintain MOI=0.2, according to the number of bacteria measured via CFU counts at each time point.

Selection and Preparation of the S. aureus Clinical Isolates for Inoculation

An S. aureus clinical isolate (CI3) was selected for in vivo experiments that was sensitive to both antibiotics to be used, insensitive to Sa87 phage on planktonic inhibition assays and could be sensitised to phage in the presence of both antibiotics in vitro. A fresh culture of CI3 on 1.5% TSA plates was prepared two days before inoculation. A single colony was resuspended in 0.9% saline (ca. 2 ml) to turbidity reading 0.5 McFarland Units and then a 1 in 100 dilution in TSB was cultured overnight one day before the in vivo experiment. CI3 pellets were harvested and resuspended in fresh TSB to reach 2.5×10⁹ Colony Forming Units (CFU)/ml.

### In Vivo Safety Study

Nine Specific Pathogen Free (SPF) Sprague Dawley rats (6 weeks old, males) were administered (n=3 rats/group) (23) 20 μl (1) Sa87 (0.5×10¹⁰ PFU/ml)+½ MIC Clindamycin in saline, (b) Sa87 (0.5×10¹⁰ PFU/ml)+½ MIC Azithromycin in saline or (3) saline. Treatments were administered intranasally twice a day for 21 days, the general wellbeing of the rat (including its appetite, behaviour, and any weight change) being closely monitored. The rats were then sacrificed and the sinus mucosal tissue, lungs, heart, liver, kidneys, spleen and brain harvested. Tissue was divided and kept fresh or 10% formalin-fixed, the latter then processed to paraffin wax, and 6 μm sections cut and stained with haematoxylin and eosin (H&E) for histopathological examination. Formalin fixed tissue was embedded in paraffin and processed to undergo H&E staining and histopathological analysis. The fresh sinus mucosa, lungs, heart, liver, kidneys, spleen, and brain were all analysed for phage titers. Faecal samples were collected from all the rats prior to the administration of the first treatment, as well as every day thereafter for phage titers. 500 μl blood samples were collected from the rat tail vein prior to the first treatment and again at 1, 2, 4 and 6 hours following the morning treatment on day 7 and 14 of treatment. This was repeated at 1, 2, 4, 6, 12, 24 and 48 hours following the last treatments on Day 21.

### Phage Titers in Faeces and Organ Tissues

1 g of faeces was suspended in 10 mL of SM buffer, centrifuged at 3214×g for 10 minutes (Eppendorf 5810 centrifuge, North Ryde, NSW, Australia), filtered (0.22 μm), and titrated for phage concentration. 1 g of rat faeces was collected from every rat at day 0 and daily after the first treatment, and directly placed into 5 mL SM buffer. Further, following killing, the rat frontal sinus mucosa was dissected and placed directly into 5 mL SM buffer. 1 cm×1 cm pieces of organ were collected from the brain, lungs, heart, liver, kidney and spleen and were also placed into 5 mL SM buffer. Phage titers and enrichment were performed as previously described (Drilling et al., 2014 supra). Briefly, organ and sinus tissues were ground in 5 mL SM buffer using a Qiagen TissueRuptor (Qiagen, Hilden, Germany). All tissue samples were then centrifuged at 3000 g for 10 minutes, filtered through a 0.22 μm of syringe filter (Pall Corporation, San Diego, CA, United States of America), and titrated for phage. 1 g of faeces was suspended in 5 mL of SM buffer, centrifuged at 3214×g for 10 minutes (Eppendorf 5810 centrifuge), filtered using a 0.22 μm filter, and titrated for phage concentration. For phage enrichment, 100 μL of 0.5 McFarland Units of S. aureus RN4220 diluted 1:100 with 10 ml TSB was grown overnight at 37° C. with shaking. Samples (1 ml tissue and faeces supernatants) were then incubated overnight with 5 ml TSB and 50 μL of S. aureus RN4220 overnight culture. The enriched samples were then centrifuged and filtered using a 0.22 μm filter, then titrated for phage. Phage titers were determined against S. aureus RN4220 using the double layer spot assay technique as described above.

### Phage Titer in the Blood Sample

500 μl of blood was collected from the rat tail vein. Blood tubes were centrifuged at 1000 rpm for 20 minutes at 4° C. The plasma was then harvested and filtered through a 0.22 μm syringe filter. Phage enrichment was then performed, as above.

### Histopathology

Rat nasal tissues were fixed in 10% neutral buffered formalin. Tissue samples were then decalcified using 9.5% nitric acid in 1% ethylenediaminetetracetic acid (EDTA), processed to paraffin wax, and 6 μm sections cut and stained with H&E. The samples were examined by a veterinary pathologist blinded to the treatments given.

### Efficacy Arm Protocol

An established murine rhinosinusitis model was used with modifications (see YU C-j et al., Chinese Journal of Otorhinolaryngology-Skull Base Surgery 6, 2011; Zhang F et al., Am J Rhinol Allergy 27 (5): 361-366, 2013; and Jin M et al., Ear Arch Oto-Rhino-L 268 (6): 857-861, 2011). Thirty Sprague Dawley rats (6 weeks old, males) underwent general anaesthesia and placement into the right nasal cavity of a 10 mm×0.5 mm×0.5 mm sponge (Gyrus ACMI, MA, United States of America), soaked in a 20 μl 0.5×10⁸ CFU S. aureus CI3 solution (sensitive to clindamycin and azithromycin but insensitive to Sa87), diluted in 0.9% saline. The sponges were placed at approximately 15 mm from the anterior nostril, located in the ostiomeatal complex. The rats were monitored for a nine-day period to allow for mucosal biofilms to form, and, on the tenth day, the sponges were removed. A two-day recovery was given to the rat before treatments begun. The rats were then randomised into one of 6 treatment groups (n=5 rats each) to receive 20 μl (1) Sa87 (0.5×10¹⁰ PFU/ml)+½ MIC clindamycin in saline, (2) Sa87 (0.5×10¹⁰ PFU/ml)+½ MIC Azithromycin in saline, (3) Sa87 (0.5×10¹⁰ PFU/ml in saline), (4) ½ MIC Clindamycin in saline, (5) ½ MIC Azithromycin in saline, and (6) saline. Treatments were administered intranasally twice a day for 14 days. Rats were monitored for weight and respiratory alterations (respiration rate; nasal discharge, hyperaemia or pruritus; and sneezing) during treatment, and, 14 days post-treatment initiation, the rats were humanely sacrificed. Their sinus mucosa was harvested for CFU counting, biofilm quantification, histopathology, and phage enumeration.

### Colony-Forming Unit (CFU) Counting of Sinus Mucosa

A fresh (0.2×0.2 cm) piece of sinus mucosa was harvested and directly placed in 250 μl 0.9% saline. The mucosa was then homogenised on the highest setting for 30 seconds, or underwent vortex mixing on the highest setting for two minutes. The homogenised supernatants were then serially diluted in 0.9% saline and spotted on sheep blood agar in triplicates (Beckton Dickenson, Franklin Lakes, NJ, United States of America) and incubated at 37° C. overnight for CFU counting of S. aureus.

### Biofilm Biomass Quantification on Sinus Mucosa

The quantification of biofilm biomass on sinus mucosa was determined using an established protocol (Singhal D et al., editors. Quantitative analysis of in vivo mucosal bacterial biofilms. International Forum of Allergy & Rhinology; 2012: Wiley Online Library (27). Briefly, the frontal sinus mucosa was harvested and placed into Dulbecco's Modified Eagle's Medium (Life Technologies, Carlsbad, CA, United States of America) on ice. One piece of mucosa from each frontal sinus was stained with a LIVE/DEAD® BacLight Bacterial Viability Kit (Life Technologies Australia). The stained mucosa was then examined at 20× magnification using a confocal laser scanning microscope (Zeiss LSM700, Carl Zeiss AG, Oberkochen, Germany). The z-stack images (21 slices, interval 1 μm) were then taken from each sample and, COMSTAT Version 2.1 software was used to measure the biomass of biofilms, the threshold setting being applied so as to minimise background staining (Heydorn A et al., Microbiology 146 (10): 2395-2407, 2000).

### Post-Treatment Sinus Microbiology Analysis

A fresh (0.2×0.2 cm) piece of mucosa from each rat sinus was harvested and homogenised in 0.9% saline. The supernatant was then harvested and transferred into a new tube without centrifugation. The collected supernatant was serially diluted and plated on blood agar plates in triplicates. After overnight incubation at 37° C., different bacterial colonies (according to the morphology and haemolytic reaction) were sub-cultured on Mannitol salt agar. Positive colonies on the Mannitol salt agar were confirmed to be S. aureus with a Staphylase Test Kit (Oxoid). Other colonies with different morphologies were further identified using MALDI-TOF (Bruker, VIC, Australia).

Post-Treatment Sinus S. aureus Phage Sensitivity

S. aureus isolates, cultured from the sinuses of rats, were sub-cultured and tested for sensitivity to phage Sa87 using the phage sensitivity test as specified above.

### Statistical Analysis

GraphPad Prism (GraphPad Prism version 8.00; GraphPad Software, La Jolla, CA) was used to graph and analyse statistical significance of data. Statistical significance for in vitro and in vivo results was computed using a one-way analysis of variance (ANOVA) or mixed ANOVA. Significance was determined at a p-value <0.05.

### Results

Synergism of Phages Combined with Antibiotics to Kill S. aureus In Vitro

Of the 17 S. aureus isolates, the two ATCC strains and 10 of the 15 CIs were sensitive or showed intermediate sensitivity to all five antibiotics tested (ie amoxicillin, vancomycin, erythromycin, clindamycin and azithromycin). A further three isolates exhibited resistance to only erythromycin, two isolates exhibited resistance to azithromycin and two exhibited multidrug resistance. The isolates were also tested for susceptibility to the three phages, J-Sa36, Sa83 and Sa87. J-Sa36 was active in vitro against only 2/17 strains tested (sensitive strains); 2/17 strains were semi-sensitive and 13/17 strains were insensitive (resistant). The in vitro activity observed for Sa83 and Sa87 was considerably higher; for Sa83, 11/17 isolates were sensitive or semi-sensitive and 6/17 insensitive and for Sa87, 12/17 isolates were sensitive or semi-sensitive and 5/17 insensitive.

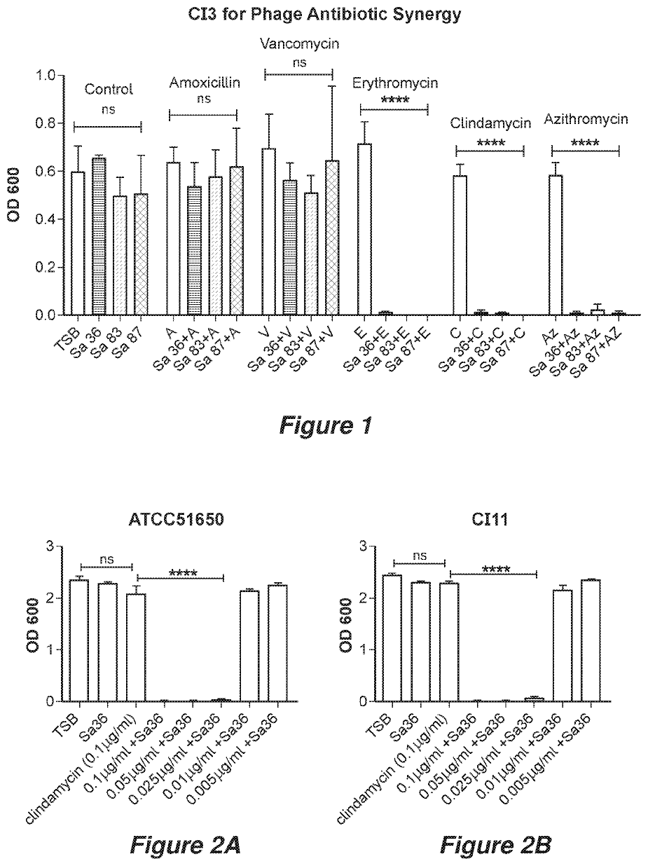

To evaluate if the combination of phage and antibiotics would be more effective than either treatment alone, the phage (ie J-Sa36, Sa83 or Sa87 (MOI=0.2)) in combination with 1/2 MIC of all 5 antibiotics against nine isolates were screened. S. aureus ATCC51650 and CIs (CI2, CI3, CI4, CI6, CI7, CI8, CI9, and CI11) were selected to represent a range of phage sensitivities. All nine isolates were either resistant (7/9) or semi-sensitive (2/9) to phage J-Sa36 alone, whilst 4/9 and 5/9 were resistant or semi-sensitive to the phages Sa83 and Sa87. A synergistic effect was defined as a statistically significant decrease in optical density (OD) that was larger than the combined decrease in OD of antibiotic and phage only controls. None of the combinations of phage with either of the cell wall actives (amoxicillin and vancomycin) showed any significant additive or synergistic effects. However, in contrast, all nine isolates showed synergism with the combination of a PSI antibiotic (erythromycin, clindamycin and azithromycin) and at least one phage. The results for one representative isolate, namely S. aureus CI3, are shown in FIG. 1.

Particularly, the combination treatment of J-Sa36 with ½ MIC clindamycin and azithromycin showed synergistic effects against six of the nine phage non-sensitive and semi-sensitive isolates, with erythromycin showing synergistic effects in five of the nine isolates. For those isolates that were insensitive to Sa83, the addition of ½ MIC of erythromycin, clindamycin and azithromycin significantly improved antimicrobial activity in three out of the four isolates screened. Further, for isolates insensitive or semi-sensitive to Sa87, the addition of ½ MIC erythromycin, clindamycin and azithromycin improved the antimicrobial activity in four out of five (for erythromycin) and all five (for clindamycin and azithromycin) insensitive and semi-sensitive isolates (see Table 2 below). As clindamycin was the most effective antibiotic in combination with all three phage types, further investigation was focused on this antibiotic.

Dose-dependence of clindamycin in combination with phage J-Sa36 to kill S. aureus J-Sa36 semi-sensitive and insensitive isolates ATCC51650 (MIC-0.2 μg/ml) and CI11 (MIC=0.2 μg/ml) were each co-incubated with J-Sa36 phage and a range of clindamycin concentrations to determine the minimum dose of clindamycin required for facilitating phage J-Sa36 anti-microbial activity. Incubation with 0.025 μg/ml (⅛ MIC), 0.05 μg/ml (¼ MIC) or 0.1 μg/ml (½ MIC) in combination with J-Sa36 equally reduced OD values compared to positive controls after 24 hours incubation (p<0.01). The lowest effective concentration of clindamycin required to facilitate phage J-Sa36 killing of ATCC51650 and CI11 was 0.025 μg/ml, ⅛th the MIC. Lower (0.01 μg/ml or 0.005 μg/ml) clindamycin concentrations combined with J-Sa36 showed no significant reduction in OD compared to either the J-Sa36 only treatment or medium only control. The results are shown in FIG. 2.

The Effect of Phage on S. aureus Biofilms with and without Clindamycin

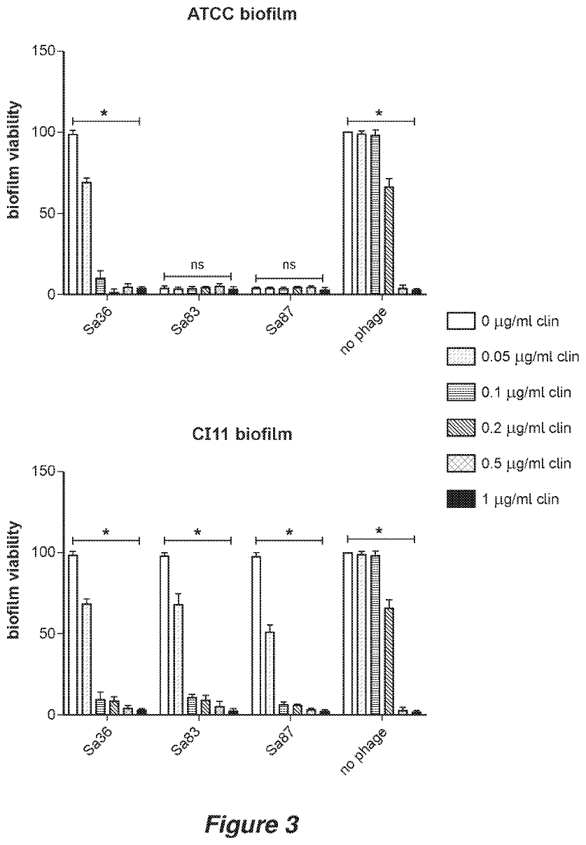

Clindamycin alone significantly reduced the viability of preformed ATCC51650 and CI11 biofilms at a concentration of 0.5 μg/ml (MIC-0.2 μg/ml) and above, with lower concentrations having no significant effect on biofilm viability. Treatment with phages J-Sa36, Sa83 or Sa87 (PFU=8×10⁶/ml) alone did not affect CI11 biofilm viability, and J-Sa36 did not affect ATCC51650 biofilm viability. However, the combination of ≥0.1 μg/ml (½ MIC) clindamycin with J-Sa36, Sa83 or Sa87 significantly reduced the viability of ATCC51650 and CI11 biofilms, compared to untreated, clindamycin only and phage only controls. The combination reduced the required concentration of clindamycin to significantly reduce biofilm viability by five-fold. The results are shown in FIG. 3.

Clindamycin-Dependent Increase in Phage Infectivity in S. aureus is Constrained in Time

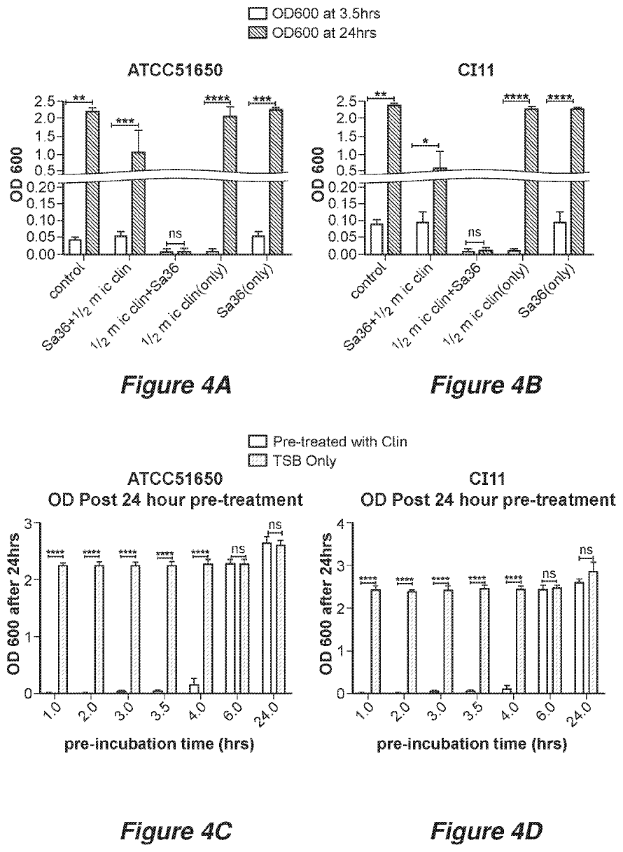

To determine if phage were sensitising S. aureus isolates to clindamycin or if clindamycin treatment was facilitating phage infection of S. aureus phage insensitive and semi-sensitive strains, both ATCC51650 and CI11 in liquid culture were incubated with either ½ MIC clindamycin or phage J-Sa36 (MOI 0.2) for 3.5 hours. Following this, the alternate treatment was added before continuous incubation with ½ MIC clindamycin and phage J-Sa36 for 24 hours. As shown in FIG. 4 (A-B), incubation with ½ MIC clindamycin or J-Sa36 only did not significantly affect bacterial viability at 24 hours. However, pre-incubation of ATCC51650 or CI11 with ½ MIC clindamycin for 3.5 hours, followed by incubation with J-Sa36 resulted in almost complete eradication of both ATCC51650 and CI11 after 24 hours. Reversing the sequence of challenge, namely incubation of the strains with J-Sa36 for 3.5 hours followed by incubation with ½ MIC clindamycin for 24 hours, resulted in a smaller but still significant reduction (p<0.01) in OD compared with untreated control. To confirm the time-dependent effects of this co-treatment, ATCC51650 or CI11 were also incubated with ½ MIC clindamycin in 10 ml TSB, then phage J-Sa36 was added into the above at different times (1h, 2 h, 3h, 3.5h, 4 h, 6 h and 24 h) (with the phage adjusted at the different time points to maintain MOI=0.2). Following 24 hours incubation, significantly reduced OD600 values were observed for cultures where J-Sa36 was added up to 4 hours post-clindamycin challenge but not when J-Sa36 was added 6 and 24 hours after challenge (see FIG. 4 (C-D)).

### Sub-Inhibitory (Below MIC Concentrations) Clindamycin Enhances Phage Activity

To determine whether clindamycin enhanced phage activity or induced an endogenous prophage, phage titers were determined from cultures of ATCC51650, CI11 and CI8 after incubation for 48 hours with ½ MIC clindamycin, phage or both. 100 μl of the resulting culture was then chloroform treated to release phage and the supernatant used to determine phage titers using ATCC51650, CI11 and CI8 as host. The initial phage inoculum of 10⁴ PFU/ml at 0 hours generated plaques only against ATCC51650 in the presence of clindamycin in the agar overlay, but no plaques were observed for CI11 or CI8. This indicates that a high MOI might be required in addition to clindamycin to facilitate phage lysis in these strains. The supernatant of the isolates exposed to ½ MIC clindamycin only for ATCC51650, CI11 and CI8 produced no plaques in any of the strains after 48 hours incubation, indicating that no intact prophages that could lyse those isolates were induced by clindamycin treatment. For the phage only treatment groups, J-Sa36 added to ATCC51650 produced no significant increase in phage titer and CI11 produced no plaques, indicating that J-Sa36 cannot infect and replicate in these isolates in the absence of clindamycin. Similar results were observed for phage Sa83; with phage only treatment of CI11 for 48 hours not producing any plaques. In contrast, co-treatment of ½ MIC clindamycin and phage led to increased phage titers in all cases. For instance, after 48 hours, ATCC51650 treated with J-Sa36 and ½ MIC clindamycin showed a concentration increase from ~10⁴ PFU/ml to 7.73±2.14×10⁶ PFU/ml. This result indicates that a clindamycin-mediated phage infection is capable of producing new phage particles, not simply leading to host cell death. The same result was observed for CI11 treated with J-Sa36 and ½ MIC clindamycin (1.9±1.3×10⁹ PFU/ml), and Sa83 and ½ MIC clindamycin (6.4±2.6×107 PFU/ml). Whilst the same trend was also observed for CI8 with Sa83 and ½ MIC clindamycin, a semi-confluent plaque was observed only at higher concentrations making quantification of titer impossible.

Sub-Inhibitory PSI Antibiotics Sensitise S. aureus Insensitive Strains to Phage In Vivo

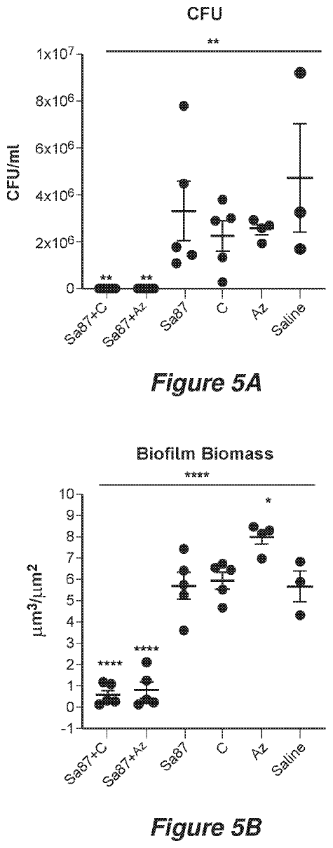

From the 30 rats across the treatment groups, 27 completed the study protocol (nb. 1 rat from the azithromycin group and 2 rats from the saline control group died due to ketamine intoxication). After two weeks of twice daily topical application of test treatments, control saline-treated rats had similar S. aureus CFU counts/ml compared to rats treated with 0.5×10¹⁰ PFU/ml Sa87, ½ MIC clindamycin or ½ MIC azithromycin (p>0.05). Sensitivity testing indicated similar sensitivity to the antibiotics and insensitivity to Sa87 for the post- and pre-treatment S. aureus isolates. In contrast, no S. aureus were cultured in rats treated with 0.5×10¹⁰ PFU/ml Sa87 in combination with ½ MIC clindamycin or azithromycin and were significantly lower than control saline-treated rats or rats treated with Sa87 or ½ MIC clindamycin or azithromycin (p=0.0086) (see FIG. 5A). However, bacterial colonies that were not S. aureus were grown from the nasal mucosa of those rats (CFU counts of <500 CFU/mL). MALDI-TOF analysis identified those bacteria to be E. coli and E. faecalis. Biofilm biomass quantitation showed similar biomass quantities in controls, Sa87 and ½ MIC clindamycin treated rats and a significant increase in biomass in ½ MIC azithromycin treated rats as compared to control. In contrast, there was a significant reduction in biofilm biomass in rats treated with 0.5×10¹⁰ PFU/ml Sa87 in combination with ½ MIC clindamycin or azithromycin compared to control (p<0.0001) (see FIG. 5B); and confocal laser microscopy of mucosa stained with a LIVE/DEAD® BacLight Bacterial Viability Kit (Life Technologies Australia) clearly revealed dead (PI, red) and live (SYTO® 9, green) biofilm associated bacteria in samples representative of each treatment group). In addition, histopathology of the maxilloturbinates by a pathologist, blinded to the treatment given, showed normal ciliated pseudostratified respiratory epithelium with no evidence of inflammation or glandular hyperplasia in rats treated with Sa87 in combination with ½ MIC clindamycin or azithromycin. In contrast, in rats treated with Sa87, ½ MIC clindamycin, ½ MIC azithromycin or saline only, there was evidence of cilial denudation and surface epithelial erosion, with desquamated necrotic luminal cellular debris admixed with neutrophils. The lamina propria was congested and oedematous and contained a more robust mixed inflammatory cell infiltrate, which also infiltrated the lining epithelium.

### Discussion

This study showed that sub-inhibitory (below MIC values) concentrations of PSI antibiotics (particularly, erythromycin, clindamycin and azithromycin) can sensitise phage non-sensitive and semi-sensitive S. aureus clinical isolates to phage infection. Sensitisation also occurred in vivo, leading to eradication of phage non-sensitive S. aureus biofilms in the presence of both antibiotics and phage.

### Example 2

The study described in this example aimed to provide further evidence that sub-inhibitory concentrations of PSI antibiotics can sensitise phage non-sensitive S. aureus to phage infection, and additionally, provide some characterisation of the progeny phage (exit phage).

### Materials and Methods

### Bacterial Strains and Growth Conditions

S. aureus strains frozen stocks were thawed and streaked on 1.5% trypticase soy agar (TSA) (Oxoid) plates at 37° C. overnight. A single colony was picked and resuspended in 2 ml 0.9% saline to achieve McFarland (McF)=0.5. 100 μl of resuspended bacterial solution was then added into 10 ml trypticase soy broth (TSB) (Oxoid) and incubated at 180 rpm for 24 hours in a 37° C. incubator. Bacteria in the late logarithmic growth phase were used for further experiments. S. aureus ATCC25923 and RN4220 were obtained from the American Type Culture Collection (ATCC) and used as reference strains or used for phage titration. S. aureus clinical isolates were identified by an independent pathology laboratory and stored at −80° C. The E. coli expression strain (E4643) was an E. coli made in Shearwin Laboratory from E4640+ (Shearwin K., School of Biological Sciences, University of Adelaide, Adelaide, SA, Australia).

### Phages and Phage Titer Determination

Two strictly lytic S. aureus phages (APTC-SA-2 and APTC-SA-12; Adelaide Phage Therapy Centre, Adelaide, SA, Australia) were used for all experiments. The phages were identified as being of the genus Kayvirus, subfamily Twortvirinae, family Herelleviridae. Phage titers were determined against S. aureus RN4220 (ATCC) using the double layer spot assay (DLSA). Briefly, 100 μl of an RN4220 overnight culture was mixed with 4 mL of 0.4% TSA and poured onto a 1.5% TSA plate. After 20 minutes, serially diluted samples of phage were spotted onto the double layer agar plates in 3 μl/spot in triplicates. Plaques were counted after overnight incubation at 37° C., and phage titers were calculated based on the dilution.

### Phage Sensitivity Test

Sensitivity tests of S. aureus clinical isolates was performed as described previously (Drilling et al., 2014 supra). Briefly, 200 μl of an overnight S. aureus culture was spread onto Columbian blood agar plates (Oxoid) and then the plates were incubated at 37° C. for 1 hour. 5 μl diluted phage (10⁶ PFU/ml) were spotted onto the plates in triplicates. Phage buffer solution (SM buffer and TSB) was spotted in the centre of the plates as a negative control. Phage sensitivity was determined as previously described after overnight incubation at 37° C. (see Zhang G et al., 2018 supra). Confluent lysis zones observed on plates were defined as phage sensitive (+), semi-confluent lysis zones were defined as phage semi-sensitive (+/−), and no plaques were defined as phage insensitive (Ooi M L et al., Jama Otolaryngol 145 (8): 723-729, 2019). Phage sensitivity was tested three times.

### Clindamycin Minimum Inhibitory Concentration (MIC) Assays

The MIC of clindamycin against the S. aureus strains was assessed using the broth microdilution technique as previously described (see Wiegand et al., 2008 supra). Results were interpreted as follows: Sensitive: ≤0.2 μg/mL; Intermediate: 0.4 μg/ml; Resistant: >0.6 μg/mL. The S. aureus strains sensitive to clindamycin were selected for further tests. The MIC was performed three times.

### Phage and Clindamycin Combination Treatment

A phage and clindamycin synergy assay was performed using 96 well plates. In brief, selected S. aureus strains (phage non-sensitive) were grown on 1.5% TSA at 37° C. overnight. A single colony was transferred to TSB to generate a culture with a starting concentration of 5×10⁵ CFU/ml. Phages were added to the bacteria culture at a MOI of 1 according to phage titers. Clindamycin was added to the cultures at ½ MIC (according to the MIC assay result). Optical density (Gorski A et al., Antibiotics (Basel) 9 (11), 2020) readings at 600 nm were recorded after 24 hours. Fresh TSB, S. aureus treated with ½ MIC clindamycin and S. aureus treated with phage at MOI=1 was used as a control.

### Exit Phage Purification

Briefly, S. aureus colonies (parent phage non-sensitive S. aureus strain) was transferred to 20 ml TSB with a starting concentration of 5×10⁵ CFU/ml. Phage (MOI=1) and ½MIC clindamycin were added. OD 600 nm were recorded after 24 hours as described above. The mixture was then centrifuged at 4,000 rpm for 10 mins to remove bacteria and filtered using a 0.2 μm syringe filter to sterilise. The exit phage titers were determined using DLSA as described above.

### Activities and Specificity of Exit Phage

The exit phage bacteriolytic activity was determined by treating parent phage non-sensitive S. aureus strains (5×10⁵ CFU/ml) with corresponding exit phages at MOI=1 at 180 rpm for 24 hours in a 37° C. incubator. OD 600 nm was recorded after 24 hours as described above. The exit phage specificity was tested by treating various S. aureus strains and the parent strain with exit phage at MOI=1 using the same procedure as described above. The exit phage lytic activity was investigated by treating parent phage non-sensitive S. aureus strains with corresponding exit phages at MOI=0.2, 0.1, 0.01 and 0.001 overnight as described above. All tests were repeated in 3 independent experiments.

### Thermal and pH Stability

All thermal and pH stability tests were performed three times according to a previous protocol with modifications (Camens S et al., Microorg 9 (9), 2021). For the thermal stability test, phages (10⁹ pfu/mL) were incubated at different temperatures (40; 50; 60; 70 and 80° C.) for 1 h. For the pH stability tests, phages (10⁹ PFU/mL) were diluted 100-fold with SM buffer with different pH values (range from 3 to 12) and incubated for 1 h at room temperature. The phage titers were then determined using DLSA as described above.

### One-Step Growth Curve

One-step growth curve was performed as detailed previously (Chang Y et al., Viruses 7 (10): 5225-5242, 2015). Briefly, 100 μL phages (1×10⁸ PFU/mL) were mixed with 1 mL S. aureus (1.5×10⁸ CFU/mL) culture at a MOI of 0.1 in 8.9 mL TSB and incubated at 37° C. for 5 min. The mixture was then centrifuged, and the pellets were resuspended in 9.9 mL fresh TSB. The resuspended pellets were incubated at 37° C. with shaking at 180 rpm. 100 μL mixture was taken every 30 min. The mixture was centrifuged at 13,000 g for 5 min, and the titres of the phage were determined using the DLSA. The experiments were carried out in triplicates.

### Measurement of Frequency of Bacteriophage-Insensitive Mutants (BIMs)

The frequency of BIMs was performed by treating an overnight culture of S. aureus (approximately 10⁹ CFU/ml) with S. aureus entry and exit phage at a MOI of 100. CaCl₂) (10 mM) and MgSO4 (10 mM) were added to the mixture at final concentration at 10 mM. Then the mixture was incubated at 37° C. for 10 min. After 10 min, the mixture left at room temperature to allow cool down. The serial diluted mixtures were plated (20 μl/spot) in triplicates and incubated overnight at 37° C. The colonies were counted and calculated. The BIM frequency was determined as surviving viable counts divided the initial viable counts. All the experiments were performed three times.

Construction of E. coli Expressing Phage Proteins and PHEARLESS Assay

A system, termed PHEARLESS (phage-based expression, amplification, and release of lytic enzyme species), was used to express phage proteins of interest in E. coli comprising three components engineered into the E. coli expression strain (E4643), namely (1) the integrated cumate inducible 186tum induction module, (2) the engineered 186Δtum prophage genome and (3) the protein expression plasmid. The E. coli containing the target protein expression plasmids were streaked out on LB agar plates with spectinomycin (50 μg/mL; Sigma Aldrich, Bayswater, VIC, Australia) and the S. aureus strains C319, C244, C330, C43, C259 and ATCC29923, were streaked out on LB agar plates. The plates were incubated at 37° C. degrees overnight. A single colony of the E. coli expression strain was picked to grow in LB broth with spectinomycin (50 μg/mL) overnight to generate a fresh bacterial culture. Similarly, a single colony of each of the S. aureus strains was picked to grow in LB broth overnight. The next day, 400 μL of the bacterial cultures was then diluted into 25 ml fresh LB broth and kept at 37° C. degrees with shaking at 180 rpm. LB agar plates with 120 μM cumate (cumic acid; Sigma Aldrich) were prepared. 200 μL of the broth culture of the target S. aureus strains (C319, C244, C330, C43, C259 and ATCC29923) was mixed with 3 ml 0.7% molten agar containing 120 μM cumate, and was poured onto the top of the LB plates. Plates were left to dry. Once the E. coli broth cultures reached OD600 nm of 0.6~0.7, the broth culture was centrifuged at 5,000 rpm for 5 min and the supernatant removed. The pellets were resuspended in fresh LB broth, serially diluted and spotted onto the target S. aureus bacterial lawn. The plates were then left to dry and incubated at 37° C. degrees overnight to allow cell lysis. The plasmid containing an “empty” protein expression construct was used as negative control.

### Results

### Phage Sensitivity Test

A total of 60 S. aureus clinical isolates were tested for their phage sensitivity in order to select those that were insensitive to both lytic APTC-SA-2 and APTC-SA-12 S. aureus phage. Among those 60 clinical isolates, four strains (C43, C319, C285 and C330) were considered phage non-sensitive since no plaques were observed. Moreover, MIC assays indicated that all four of these strains were sensitive to clindamycin. ATCC25923 was used as a reference control strain and was sensitive to both phage strains and to clindamycin. The results of the phage and clindamycin sensitivity tests are shown in Table 3.

TABLE 3
Phage host range and antibiotic sensitivity

| | APTC- | APTC- | Clindamycin MIC |
|---|---|---|---|
| | SA-2 | SA-12 | (μg/ml) |
| C43 | − | − | 0.2 |
| C330 | − | − | 0.2 |
| C285 | − | − | 0.2 |
| C319 | − | − | 0.2 |
| ATCC25923 | + | + | 0.2 |

+ = phage sensitive; − = phage resistant.

Phage Non-Sensitive S. aureus is Sensitised to Phage by Low Concentration Clindamycin

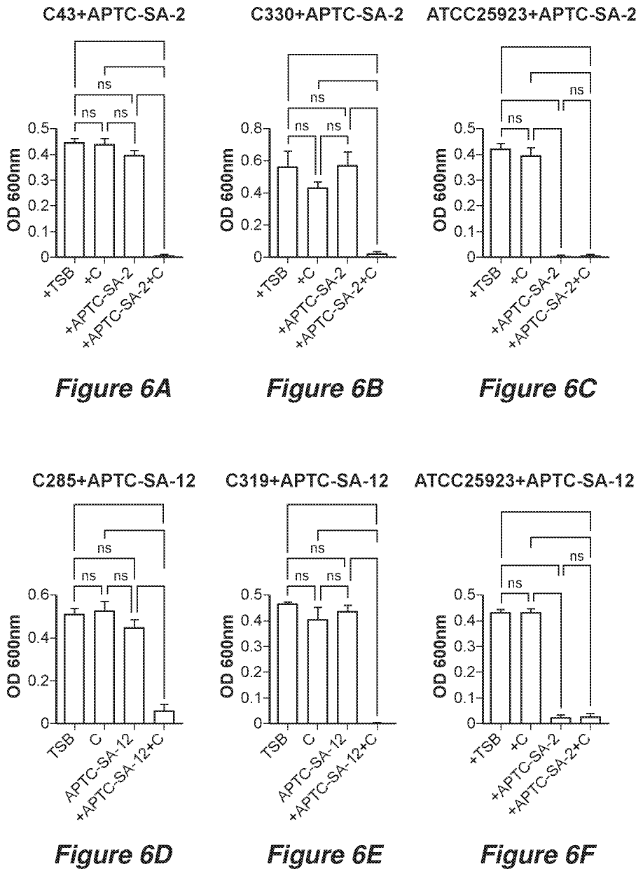

Four phage non-sensitive S. aureus strains (C43, C330, C285 and C319) were selected to be treated with the APTC-SA-2 and APTC-SA-12 phage combined with ½ MIC clindamycin. The combination treatment could kill the bacteria after 24 hours and was significantly more effective than control (ie bacteria with TSB), or treatment with ½ MIC clindamycin and phage only. The growth of S. aureus bacteria was similar among control, ½ MIC clindamycin treated, and phage only treated groups (see FIG. 6A-B, D-E). ATCC 25923 was sensitive to both phage types, and phage in combination with ½ MIC clindamycin were equally effective at killing this strain compared to no-treatment control and to ½ MIC clindamycin (FIGS. 6C and F).

### Exit Phage Bactericidal Activity

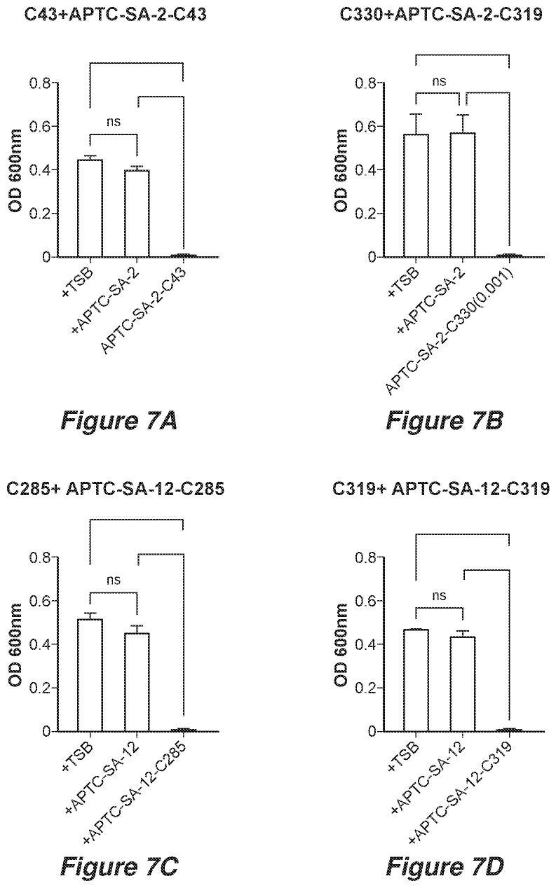

To evaluate whether the progeny phage particles (exit phage), produced after sensitisation of non-sensitive S. aureus strains with ½ MIC clindamycin and adding the entry phage (APTC-SA-2 and APTC-SA-12), were effective at killing their own parent strain, exit phage particles were purified for both APTC-SA-2 or APTC-SA-12 entry phage after they went through an infection cycle of two representative non-sensitive S. aureus strains. Compared with positive control (TSB) and entry phage treatment (APTC-SA-2 or APTC-SA-12), it was found that the exit phage could significantly reduce the viability of parent strains in the absence of ½ MIC clindamycin at MOI=1 (FIG. 7).

### Exit Phage Specificity and Lytic Activity

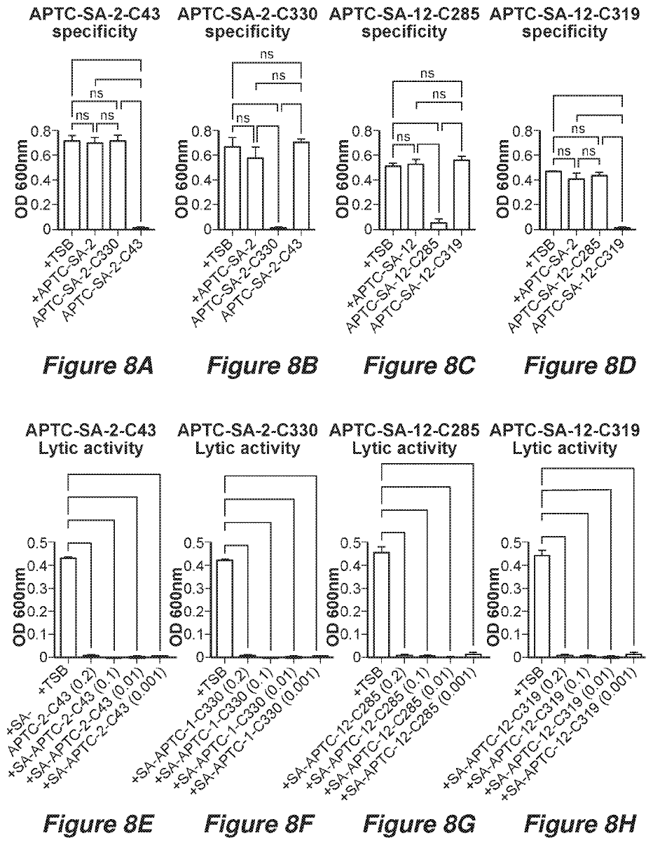

To further investigate the specificity of the exit phage, the exit phage APTC-SA-2-C43, APTC-SA-2-C330, APTC-SA-2-C285, and APTC-SA-2-C319 were used to treat phage non-sensitive S. aureus clinical isolates C43, C330, C285, and C319 at MOI=1. The results, shown in FIG. 8A-D, indicated that the exit phages were active against their parent strain but not against any of the other isolates. Next, the lytic activity of the exit phage was tested (and compared to the entry phage), and this showed that the exit phage could infect the parent strain at a MOI as low as 0.001. Moreover, CFU counts showed the presence of <67 CFU/mL, indicating S. aureus eradication when the parent strain was treated with the exit phage at MOI as low as 0.001.

### Exit Phage Characterisation

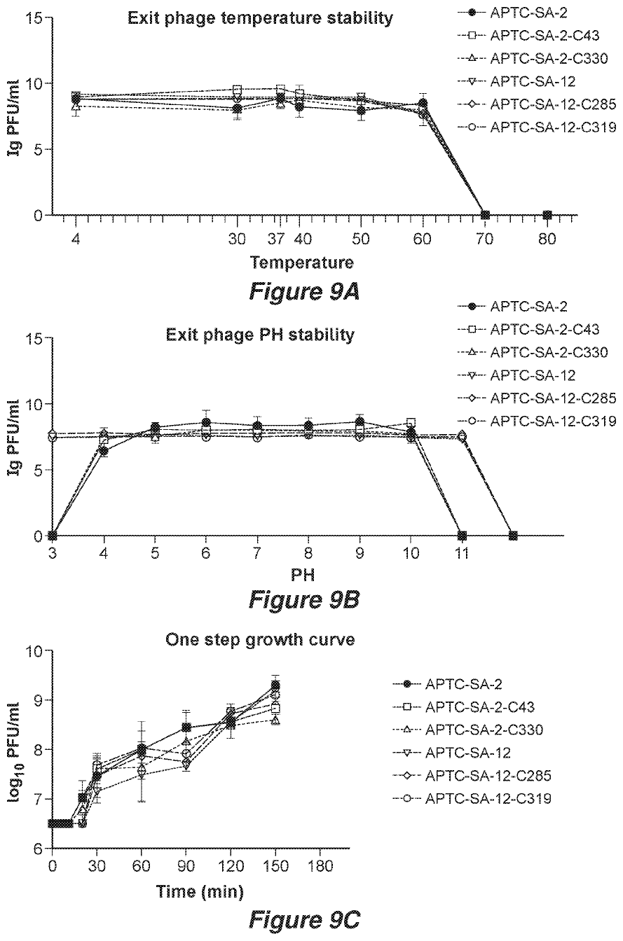

The phage temperature stability, pH stability and one-step growth curve for entry and exit phages were evaluated and compared. In comparison with APTC-SA-2, the exit phage APTC-SA-2-C43 and APTC-SA-2-C330's temperature stability and pH stability were similar. With the one-step growth curves, it was found that the entry phage and exit phage showed similar latency time and burst size (FIG. 9A-C). Similar results were noted when comparing the results obtained from the entry phage APTC-SA-12 with the exit phage APTC-SA-12-C285 and APTC-SA-12-C319.

The frequency of BIMs of entry phage and exit phage have been determined. Results showed the frequency of BIM generation by the exit phage was lower compared with their corresponding entry phage (Table 4).

TABLE 4
Frequency of emergence of phage insensitive mutants
(BIMs) of entry phage and exit phage

| Phage | *BIM frequency± (Mean SD) | |
|---|---|---|
| APTC-SA-2 | 0.26e−07 | 0.21e−07 |
| APTC-SA-2-C43 | 0.08e−07 | 0.08e−07 |
| APTC-SA 2 C330 | 0.07e−07 | 0.07e−07 |
| APTC-SA-12 | 0.17e−07 | 0.12e−07 |
| APTC-SA-12-C285 | 0.03e−07 | 0.03e−07 |
| APTC-SA-12-C319 | 0.04e−07 | 0.04e−07 |

*Experiment performed using manufacturing strain C16 (S. aureus). The average of three independent experiments is shown.

### PHEARLESS Assay

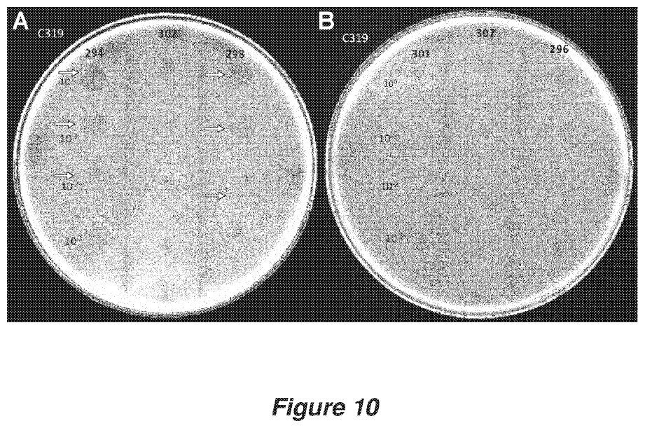

The PHEARLESS assay described above was conducted to investigate the lytic activity of the exit phage. In particular, the assay was conducted to investigate and compare the lytic activity of the lysin protein of the entry phage (LysK) with that of the exit phage (denoted mLysK). Briefly, exit phage mLysK expression constructs were prepared from APTC-SA-2-C330 and from APTC-SA-2-C319 and an entry phage LysK expression construct was prepared from APTC-SA-2. All expression constructs were transformed into the PHEARLESS system and tested against the S. aureus phage non-sensitive strains C319, C285, C330 and C43 and also against the phage sensitive ATCC25923 to evaluate their ability to facilitate bacterial lysis. The entry phage LysK only showed weak lytic activity against ATCC25923 as a zone of clearing was observed at the highest dosage only, whilst all other strains remained unaffected by the entry phage LysK. In contrast, the expressed exit phage mLysK from APTC-SA-2-C330 (298) and from APTC-SA-12-C319 (294) showed lytic activity against all of the tested strains in a dose-dependent way (zone of clearing appearance clear to blurred from highest dosage to lowest dosage). Images of representative plates showing the lytic activity of APTC-SA-2-C330 (298) and from APTC-SA-12-C319 (294) on S. aureus strain C19 is shown in FIG. 10.

These results demonstrate that the exit phage contained a modified lysin protein, and as such the exit phage encodes an endopeptidase with modified activity and/or target specificity. Further, sequencing of the LysK gene from the exit phage demonstrated that the exit phage contained at least a mutation in the lysin gene which produces the modified lysin protein.

### Discussion

To date no strategies have been reported that can sensitise S. aureus and MRSA phage resistant strains to phage. Consequently, patients infected with such strains are excluded from phage therapy with few or no therapeutic options remaining to treat those often-devastating infections. Here, it was found that two lytic S. aureus phages, when used to treat four non-sensitive S. aureus clinical isolates in combination with ½ MIC clindamycin, could significantly reduce the growth of the insensitive S. aureus clinical isolates. Further, it was found that the harvested and purified exit phage were able to re-infect the parent S. aureus strains (only), without the help of clindamycin, and with at least a 1000-fold higher level of lytic activity. Together with characterisation that showed that the stability and one-step growth curve of the exit phage showed no significant difference with the corresponding entry phage, these results indicate that these exit phage could be useful in treating infections of phage insensitive S. aureus strains in patients.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be readily appreciated by those skilled in the art that methods, phage and phage proteins of the present disclosure are not restricted in their use to the particular application described. Neither are the methods, phage and phage proteins restricted in their preferred embodiment(s) with regard to the particular elements and/or features described or depicted herein. Further, it will be readily appreciated that the methods, phage and phage proteins are not limited to the embodiment(s) disclosed, but are capable of numerous rearrangements, modifications and substitutions without departing from the scope of the present disclosure.

1. A method of producing a modified phage, said method comprising the steps of:
(i) treating a host bacteria insensitive to a selected phage strain with a suitable amount of the said phage strain and at least one antibacterial agent comprising protein synthesis inhibitory activity, and culturing the bacteria for a period and under conditions suitable for the phage to infect the bacteria and replicate to generate progeny phage; and
(ii) recovering and expanding progeny phage which are phage with a modified capability to infect and cause lysis of said host bacteria.

2. A method according to claim 1 for producing a modified phage for use in a phage therapy of an infection in a subject, wherein said method comprises the steps of:
(i) treating a host bacteria that has been isolated from the said subject and which is insensitive to a selected phage strain with a suitable amount of the said phage strain and at least one antibacterial agent comprising protein synthesis inhibitory activity, and culturing the bacteria for a period and under conditions suitable for the bacteriophage to infect the bacteria and replicate to generate progeny phage; (ii) recovering and expanding progeny phage which are phage with a modified capability to infect and cause lysis of said host bacteria; and
(iii) providing the modified phage of step (ii) in a pharmaceutical composition for administration to the said subject.

3. A method according to claim 1, wherein the host bacteria is selected from the group consisting of Staphylococcus species, Streptococcus species, Enterococcus species, Corynebacterium species, Bacteroides species, Bacillus species, Yersinia species, Legionella species, Francisella species, Salmonella species, Mycobacterium species, Acinetobacter species, Clostridium species, Pseudomonas species, and Entobacteriaceae species, or is selected from the group consisting of S. aureus, S. epidermidis, S. lugdunensis, Acinetobacter baumannii, Clostridium difficile, and Pseudomonas aeruginosa.

4. (canceled)

5. A method according to claim 1, wherein the host bacteria is an antibiotic-resistant bacteria, or a multidrug-resistant (MDR) bacteria.

6. (canceled)

7. A method according to claim 1, wherein the host bacteria is a clinical isolate, or a clinical isolate from a bacterial infection characterised by the presence of a bacterial biofilm.

8. (canceled)

9. A method according to claim 1, wherein the host bacteria is treated in step (i) with a phage strain (entry phage) selected from a phage bank.

10. A method according to claim 1, wherein the at least one antibacterial agent comprising protein synthesis inhibitory activity is provided at an amount that is sub-inhibitory.

11. A method according to claim 1, wherein the antibacterial agent is selected from one or more of the group consisting of erythromycin, clindamycin and azithromycin.

12. A method according to claim 1, wherein step (ii) comprises two or more steps of culturing the host bacteria in the presence of the progeny phage (exit phage) to expand the phage number.

13. A method according to claim 1, wherein the modified phage of step (ii) show an enhanced level of lytic activity against the host bacteria that is at least 500-fold higher than that of the selected entry phage.

14. A method of treating a bacterial infection in a subject, said method comprising the steps of: 1. obtaining a sample of a bacteria causative of an infection in said subject,
2. treating the bacteria with a suitable amount of a selected phage strain to which the bacteria is insensitive and at least one antibacterial agent comprising protein synthesis inhibitor activity, and culturing the bacteria for a period and under conditions suitable for the bacteriophage to infect the bacteria and replicate to generate progeny phage;
3. recovering and expanding progeny phage which are phage with a modified capability to infect and cause lysis of said bacteria; and
4. administering to the subject an effective amount of the modified phage of step 3, or a further propagated form thereof, to treat the infection.

15. A method according to claim 14, wherein the host bacteria is selected from the group consisting of Staphylococcus species, Streptococcus species, Enterococcus species, Corynebacterium species, Bacteroides species, Bacillus species, Yersinia species, Legionella species, Francisella species, Salmonella species, Mycobacterium species, Acinetobacter species, Clostridium species, Pseudomonas species, and Entobacteriaceae species, or is selected from the group consisting of S. aureus, S. epidermidis, S. lugdunensis, Acinetobacter baumannii, Clostridium difficile, and Pseudomonas aeruginosa.

16. (canceled)

17. A method according to claim 14, wherein the bacteria causative of the infection is an antibiotic-resistant bacteria, or is a multidrug-resistant (MDR) bacteria.

18. (canceled)

19. A method according to claim 14, wherein the bacterial infection is characterised by the presence of a bacterial biofilm.

20. A method according to claim 14, wherein the bacteria is treated in step 2 with a phage strain (entry phage) selected from a phage bank.

21. A method according to claim 14, wherein the at least one antibacterial agent comprising protein synthesis inhibitory activity is provided at an amount that is sub-inhibitory.

22. A method according to claim 14, wherein the antibacterial agent is selected from one or more of the group consisting of erythromycin, clindamycin and azithromycin.

23. A method according to claim 14, wherein step 3 comprises two or more steps of culturing the bacteria in the presence of the progeny phage (exit phage) to expand the phage number.

24. A method according to claim 14, wherein the modified phage of step 3 show an enhanced level of lytic activity against the bacteria that is at least 500-fold higher than that of the selected entry phage.

25. A method according to claim 14, wherein the modified phage of step 4, or a further propagated form thereof, are provided as a pharmaceutical composition to treat the infection.

26. A method according to claim 14, wherein the modified phage, or a further propagated form thereof, are used in a combination therapy with a suitable antibacterial agent to treat the infection.

27. A method according to claim 14, wherein the bacterial infection is associated with a sinus infection, a skin ulcer, a skin infection, or a lung infection.

28.-34. (canceled)

35. A method according to claim 2, wherein the host bacteria is selected from the group consisting of Staphylococcus species, Streptococcus species, Enterococcus species, Corynebacterium species, Bacteroides species, Bacillus species, Yersinia species, Legionella species, Francisella species, Salmonella species, Mycobacterium species, Acinetobacter species, Clostridium species, Pseudomonas species, and Entobacteriaceae species, or is selected from the group consisting of S. aureus, S. epidermidis, S. lugdunensis, Acinetobacter baumannii, Clostridium difficile, and Pseudomonas aeruginosa.
